(19) United States
(12) Patent Application Publication
(10) Pub. No.: US 2025/0037808 A1
(43) Pub. Date: Jan. 30, 2025

(54) A Privacy Enabled System And Method For Managing Logistics For Clinical Study Participants

(71) Applicant: Gray Consulting, Inc., Philadelphia, PA (US)

(72) Inventors: WILLIAM WING, San Francisco, CA (US); SCOTT GRAY, Fort Lauderdale, FL (US); TREVOR SMITH, Salt Lake City, UT (US); BRENT SNYDER, Souderton, PA (US); DONNA BRUMBACK, Raleigh, NC (US)

(21) Appl. No.: 18/278,203

(22) PCT Filed: Oct. 31, 2022
(86) PCT No.: PCT/US2022/048487

§ 371 Date: Aug. 22, 2023

Related U.S. Application Data
(63) Continuation-in-part of application No. PCT/US2022/021443, filed on Mar. 22, 2022.

(51) Int. Cl. G16H 10/20; G16H 40/20

(52) U.S. Cl. CPC G16H 10/20; G16H 40/20

(57) ABSTRACT

Systems for visualization and management of a clinical study have a processor, an input device, a database storing data related to the clinical study, and a visit schedule interface. The data have a study visit schedule. The study visit schedule has nodes and a track having a step sequence. Each node has a node position and node attributes. The node position has a node track identifying the track and a node step. The data may also have a visit travel policy and the node attributes may have a visit policy label associated with the visit travel policy.

### CROSS REFERENCE TO RELATED APPLICATIONS

This application claims the benefit of U.S. Provisional Patent Application 63/281,046, filed Nov. 18, 2021, and is continuation-in-part of Patent Cooperation Treaty Application PCT/US22/21443, filed Mar. 22, 2022, both of which are hereby incorporated by reference in their entirety.

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

The present invention relates to a system for managing participants in a clinical study. Clinical studies may be organized, funded, or sponsored by various entities, or sponsors, (e.g., medical or pharmaceutical companies, government agencies, hospitals, doctors, health care providers, and others). Sponsors may set various requirements for the study, including for example, the time, length, schedules, and goals of the study; the drugs or medications to be studied and their dosages; the treatments or procedures to be studied and their duration; the locations where to perform procedures and tests; the types and numbers of study participants, rules and policies for the management and recruitment of study participant, and various other rules and requirements. Sponsors may also establish the study budgets and financial rules.

Clinical studies can be expensive, with substantial budgets for identification, recruitment, and management of volunteer study participants. When participants drop out of a study, it may lead to substantial additional costs, including due to replacements, delays, duplications, and various other reasons. Therefore, reducing the number of participants who drop out of a clinical study may lead to a reduction, or more efficient use, of study budgets.

Participants may drop out of studies for various reasons, including difficulties traveling to study locations, financial challenges, scheduling interference with work or family commitments, and others. Patients (“participants” and “patients” are used interchangeably below) in a clinical study may have health conditions that constrain their abilities and availability. It is therefore desirable to provide a way of assisting patients by unburdening them and alleviating study participation challenges. One way may be by providing streamlined methods (e.g., apps, software, website, call centers, etc.) that participants can use at their convenience for participation related tasks, such as booking travel, expense reimbursement, and others. Such solutions that may allow participants to, for example, arrange their own travel, still demand time and effort from participants. Also, for privacy, security, consistency, simplicity, and various other reasons, participant facing systems may need to enforce the same study wide travel and financial policies for all participants who book their own travel, making it difficult to apply individualized travel policies. Embodiments of the present invention enable an efficient and automated concierge type service allowing travel policy individualization for each participant and reducing participants' time spent managing their participation in the clinical study.

Additionally, systems that collect, store, process, access, or display, personal or protected health information must implement strict privacy, data security, and auditing measures compliant with multi-jurisdictional privacy and data security laws, and regulations. Various national and international legal frameworks (e.g., HIPAA and HITECH in the USA; GDPR in the EU, privacy laws in the UK, Russia, Turkey, Canada, Australia, and many others) (“Privacy Laws”) establish complex standards intended to guard various information related to individuals, including Personal Information (PI), personally identifiable information (PII), personal data (PD), protected health information (PHI) (including GDPR's, “Data concerning health” or “Health Records”).

For consistency and ease of reference, below “Personal Information” or “PI” are used to refer to all types of sensitive personal information, including PII, PD, Health Records and PHI, that may be regulated by data privacy law or regulation in any jurisdiction.

### BRIEF SUMMARY OF THE INVENTION

Embodiments of the present invention are related to systems to facilitate creation, digitization, modification, visualization and management of clinical studies and clinical study participants. In an embodiment of the present invention a system for visualization and management of a clinical study comprises a processor, an input device, a database storing data related to the clinical study, and a visit schedule interface. The data comprise a study visit schedule. The study visit schedule comprises a plurality of nodes and a track having a step sequence. Each node of the plurality of nodes comprising a node position and node attributes. The node position comprises a node track identifying the track and a node step. In an embodiment of the invention, the data may also comprise a visit travel policy and the node attributes may comprise a visit policy label associated with the visit travel policy.

The plurality of nodes are ordered in the step sequence according to the node step of each node. The track is selected from a study track, an exit track, and an unscheduled visits track. The visit schedule interface may comprise a visit schedule control, a selection control, and an exit track control. The visit schedule interface receives the visit schedule from the database and displays a track graphic comprising a plurality of selectable node graphics, each of the selectable node graphics associated with one node of the plurality of nodes. The track graphic visualizes the step sequence. Each of the selectable node graphics comprises visual attributes representative of node attributes. The visit schedule interface may be configured to enable use of the selection control to select one or more nodes from the plurality of nodes as selected nodes by selecting one or more of the selectable node graphics. The selected nodes comprise a selection position.

In other embodiments the visit schedule interface is configured to enable using the visit schedule control to add a new node to the visit track, to delete one or more nodes of the plurality of nodes from the visit schedule, to move one or more nodes of the plurality of nodes to a move to position, and to modify the node attributes of one or more nodes of the plurality of nodes. The visit schedule interface, responsive to using the visit schedule control to add a new node is configured to receive a new node position and new node attributes and cause the database to store the new node into the visit schedule inserting the new node into the step sequence according to the new node position. The new node position and new node attributes may be based on the selection position. The visit schedule interface, responsive to using the visit schedule control to delete one or more nodes configured to cause the database to delete the one or more nodes from the visit schedule. The deleted one or more nodes may be the selected nodes. The visit schedule interface, responsive to using the visit schedule control to move one or more nodes, is configured to receive the move to position and to cause the database to store the one or more nodes to be moved in the visit schedule updating the step sequence according to a move from position and the move to position. The one or more nodes to be moved may be the selected nodes and the move from position may be the selection position. Responsive to using the visit schedule control to add a new node, delete one or more nodes, move one or more nodes, and modify node attributes of one or more nodes, the visit schedule interface is configured to update the track graphic to visualize the added new nodes, deleted nodes, moved nodes, and modified node attributes.

Another embodiment may comprise one or more exit track, and the visit schedule interface may be configured to receive from the exit track control one or more of an indication to add one or more new exit tracks, an indication to delete one or more of the exit tracks, and, a modified exit track name and modified exit reasons for the one more of the exit tracks. Responsive to receiving the indication to add a new exit track, the visit schedule interface is configured to cause the database to generate and store the new exit track in the visit schedule, and to display a new exit track graphic visually representative of the new exit track. Responsive to receiving the indication to delete one or more of the exit tracks, the visit schedule interface is configured to cause the database to delete the deleted exit tracks from the visit schedule, and to stop displaying the exit track graphics associated with the deleted exit tracks. Responsive to receiving the modified exit track name and modified exit reasons the visit schedule interface is configured to cause the database to store the modified exit track name and modified exit reasons in the visit schedule replacing the exit track name and the exit reasons, and to update the exit track graphic to be visually representative of the modified exit track name and modified exit reasons.

### BRIEF DESCRIPTION OF THE SEVERAL VIEWS OF THE DRAWING

The advantages and features of the present invention will be better understood as the following description is read in conjunction with the accompanying drawings, wherein:

FIG. 1 is a diagram illustrating an embodiment of the present invention.

FIG. 2 is a diagram illustrating a database embodiment in an embodiment of the present invention.

FIG. 3 is a diagram illustrating a flowchart of an embodiment of the present invention.

FIG. 4 is a diagram illustrating a flowchart of an embodiment of the present invention.

FIG. 5 is a diagram of an embodiment of a clinical study graphical user interface in an embodiment of the present invention.

FIG. 6 is a diagram of an embodiment of a clinical study graphical user interface in an embodiment of the present invention.

FIG. 7 is a diagram of an embodiment of a graphical user interface of an embodiment of the present invention.

FIG. 8 is a diagram of an embodiment of a clinical study graphical user interface in an embodiment of the present invention.

FIG. 9 is a diagram of an embodiment of a clinical study graphical user interface in an embodiment of the present invention.

FIG. 10 is a diagram of an embodiment of a clinical study participant graphical user interface in an embodiment of the present invention.

FIG. 11 is a diagram of an embodiment of a clinical study participant graphical user interface in an embodiment of the present invention.

FIG. 12 is a diagram of an embodiment of a clinical study participant travel profile graphical user interface in an embodiment of the present invention.

FIGS. 13-26 are diagrams illustrating embodiments of travel profiles of a clinical study participant in an embodiment of the present invention.

FIG. 27 is a diagram of an embodiment of a clinical study participant travel graphical user interface in an embodiment of the present invention.

FIG. 28 is a diagram illustrating an embodiment of an expenses interface in an embodiment of the present invention.

FIG. 29 is a diagram of an embodiment of a clinical study sites graphical user interface in an embodiment of the present invention.

FIG. 30 is a diagram of an embodiment of a clinical study visit schedule graphical user interface in an embodiment of the present invention.

FIG. 31 is a diagram of an embodiment of a clinical study participant visit schedule graphical user interface in an embodiment of the present invention.

FIGS. 32-35 are diagrams of embodiments of clinical study participant travel graphical user interface in an embodiment of the present invention.

FIG. 36 is a diagram of an embodiment of a travel planning graphical user interface in an embodiment of the present invention.

FIG. 37 is a diagram of an embodiment of a clinical study participant travel policies graphical user interface in an embodiment of the present invention.

FIG. 38 is a diagram of an embodiment of a clinical study travel policies graphical user interface in an embodiment of the present invention.

FIG. 39 is an illustration of an embodiment of a clinical study travel policy in an embodiment of the present invention.

FIG. 40 is a diagram of an embodiment of a visit schedule interface of an embodiment of the present invention.

FIG. 41 is a diagram of an embodiment of a visit schedule interface of an embodiment of the present invention.

FIG. 42 is a diagram of an embodiment of a visit schedule interface control of an embodiment of the present invention.

FIG. 43 is a diagram of an embodiment of a visit schedule interface of an embodiment of the present invention.

FIG. 44 is a diagram of an embodiment of a visit schedule interface of an embodiment of the present invention.

FIG. 45 is a diagram of an embodiment of a visit schedule interface of an embodiment of the present invention.

FIG. 46 is a diagram of an embodiment of a visit schedule interface of an embodiment of the present invention.

FIG. 47 is a diagram of an embodiment of a visit schedule interface of an embodiment of the present invention.

FIG. 48 is a diagram of an embodiment of a visit schedule interface of an embodiment of the present invention.

FIG. 49 is a diagram of an embodiment of a visit schedule interface of an embodiment of the present invention.

FIG. 50 is a diagram of an embodiment of a visit schedule interface of an embodiment of the present invention.

For clarity all reference numerals may not be included in every figure.

### DETAILED DESCRIPTION OF THE INVENTION

The present invention is related to the management of individuals, and their associated personal information, participating in a broad range of experimental or observational medical, health, sociological, and other scientific research or studies, collectively referred to below as clinical study 25. While the present disclosure refers to clinical studies to illustrate embodiments of the invention, the invention should not be understood to be limited to only clinical studies.

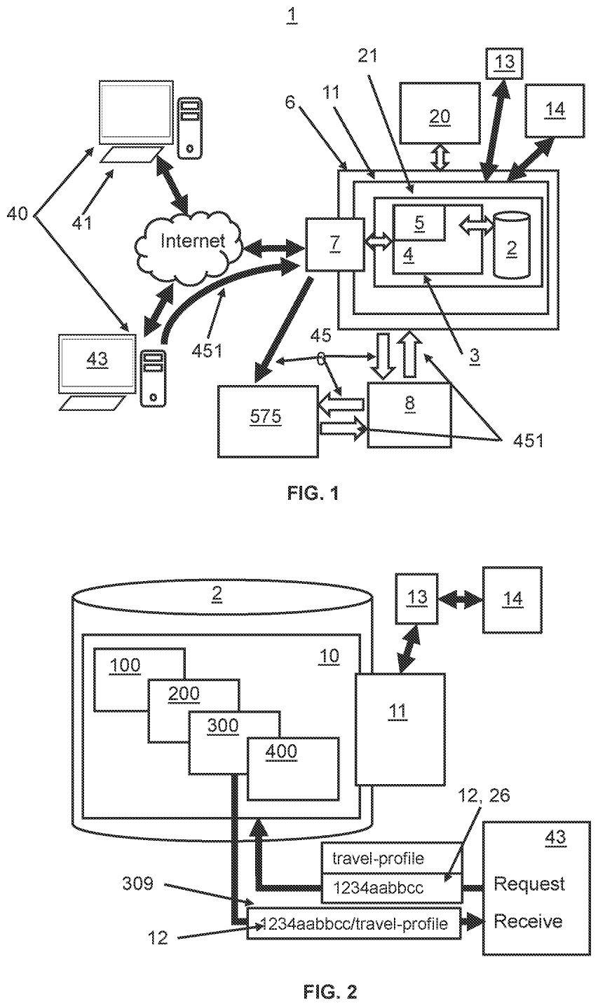

FIG. 1 illustrates a system according to a preferred embodiment of the present invention, which may be implemented as a distributed, networked, system 1 utilizing a database 2, server 3, private cloud 6, and user workstations 40. System 1 may further utilize an internet gateway 7, monitoring tools 20, and data encryption tools 11.

Database 2 may be a SQL, Non-SQL, relational, or non-relational database (e.g., MySQL, Oracle, Mongo, Cassandra, ElasticSearch, Neo4J, and others) or any other datastore or repository for persistently storing and managing collections of data according to the invention. Database 2 may comprise a database server comprising a processor. Preferably, database 2 is secured within the private cloud 6, and may be further secured by isolating it in a private subnet within the private cloud 6, effectively preventing anyone outside the private cloud from gaining access to database 2. All data stored in database 2 preferably are encrypted while at rest (e.g., when stored in database 2) using encryption tools 11. Data 10 stored in database 2 preferably are further protected, and/or obfuscated, for example by associating data records with randomly generated unique identifiers 12 (e.g., uuid, guid, others) and using the unique identifiers 12 to query, access, and/or reference records or portions of data 10. Unique identifiers 12 allow data 10 to be deidentified or pseudonymized. For example, in a SQL database 2, unique identifier 12 may be used as a primary table key, or in a lookup table associating unique identifier 12 with data or even a datum. For non-SQL/non-relational database 2, the unique identifier may be used to access records or values in, for example, as a key or document identifier, within nodes/vertices, in key value pairs, and other methods that use unique identifier 12 instead of referencing actual data values. Other methods of using unique identifier 12 and other methods of protecting data in relational and non-relational databases are well known. Servers 3 may be web servers 4, application servers 5, or both, and may use dedicated or shared computing resources (e.g., processors, hardware, etc.). Servers 3 preferably are secured within private cloud 6.

Workstation 40 may use display 42 to display data received from database 2 or from input device 41. Workstation 40 may comprise a graphical user interface (GUI) 43 that visualizes and facilitates the input and manipulation of data by a user using workstation 40 and input device 41. User may be any person authorized to access system 1 through workstation 40, and who may be associated with a study, such as staff affiliated with study sponsors, CROs, clinical sites, institutions, vendors, participants, and in general any person who is. Workstation 40 may be any computing device comprising a processor such as a personal computer, laptop, tablet, mobile device, thin client, or any other device capable of displaying the GUI and connecting to a network (e.g., the internet, internal networks, other public or private networks). Input device 41 may be a mouse, keyboard, microphone, voice, optical input, camera, tape, and other known devices or methods for inputting data.

Display 42 may be any display or monitor (e.g., screen, projector, e ink, holographic, etc.) and a display controller (e.g., display hardware and software controlling the display), as well as any other hardware or software instrumentality, or interface used or known to properly operate display 42. Display 42 may display GUI 43 using client software or program capable of visualizing information received over a network and/or input device 41, and of securely transmitting the information over a network, for example a browser capable of utilizing network protocols (e.g., HTTP, HTTPS, AS2, AS3, AS4, RTP, UDP, etc.) to send and receive data over networks and of displaying data using markup (e.g., HTML, XML, SGML, etc.) or other visualization techniques. In embodiments with more than one workstation 40, each workstation 40 and display 42 may display different data, and different aspects and/or views of GUI 43 permitting different users to simultaneously perform different actions.

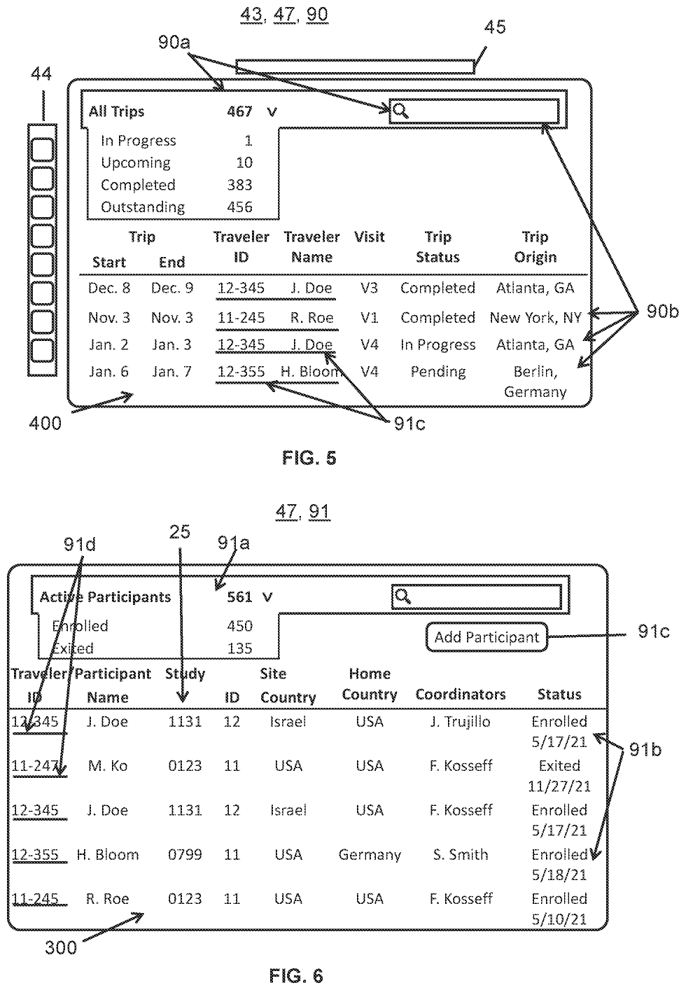

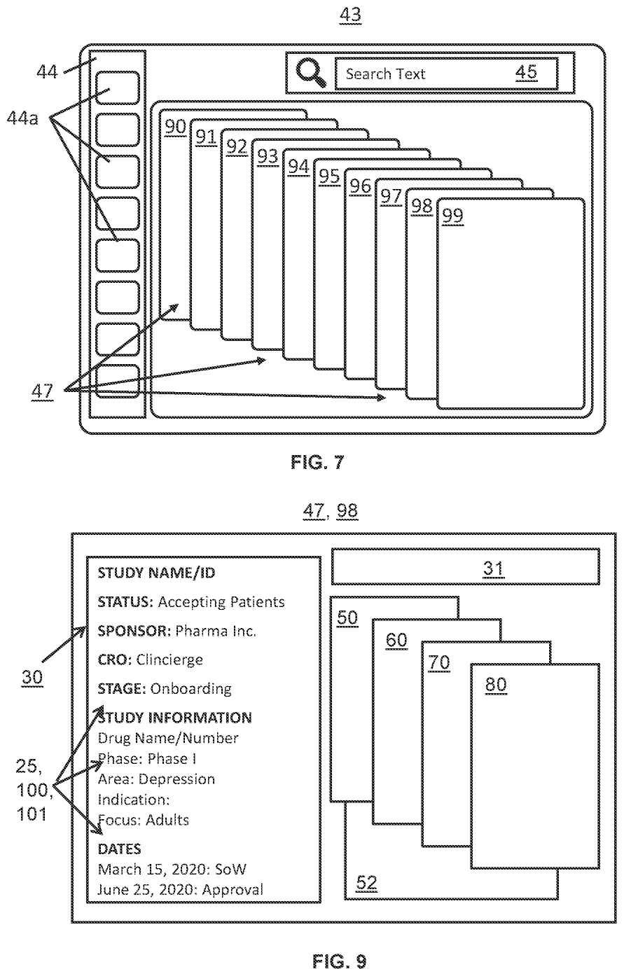

An embodiment of GUI 43 illustrated in FIG. 7, facilitates the efficient input and customization of data as well as efficient navigation through multiple visual panels and views of data 10, configured to efficiently facilitate different operations, such as entry, modification, and visualization of participant data, clinical site data, travel policies data, user security assignments, visit schedules, travel booking and travel management (including transportation and accommodations), expense management and reimbursement, and various others. The graphical user interface 43 comprises a control panel 44, main search control 45, and interface 47. In one embodiment, GUI 43 is configured to enable a user to utilize control panel 44 and controls 44a to access interface 47. Embodiments of interface 47 illustrated in FIGS. 5-9, may comprise various views and/or panels, including trips 90, travelers 91, available studies 92, study 98, available sites 93, site 99, coordinators 94, institutions 95, available countries 96, and country 97.

FIG. 7 illustrates an embodiment of GUI 43, control panel 44, main search control 45, and interface 47 are configured to enable efficient navigation between the different panels, views, and interfaces by utilizing (e.g., selecting, clicking, touching, pointing, typing, etc.) various controls (e.g., icon, tab, button, pull down list, menu, search input, clickable links etc.). For example, GUI 43 may be configured to so that the assignments interface may be accessed (e.g., displayed and switched to) by utilizing an assignments control, participants interface 50 may be accessed with control 51, study sites interface 60 may be accessed with control 61, coordinators interface 94 may be accessed with control 94a, travel policies interface 70 may be accessed with control 71, and visit schedule interface 80 may be accessed by utilizing visit schedule navigation control 81.

Interface 47 may be configured to display various interactive views and panels to permit a user to efficiently provide services for participants, including selecting and booking travel itineraries (e.g., flights, ground transportation, hotels, housing), and providing notes, instructions, and directions for participants and/or travel service providers. Interface 47 of GUI 43 also may be configured to enable input, customization, and/or correction of participant dataset 300, to enter new information, correct existing information, or modify and supplement participant dataset 300 to reflect individualized requirements and preferences of a participant, for example enabling a participant specific travel policies if warranted. GUI 43 may be configured to enable such input, customization, or correction of participant dataset 300 during the provision of concierge type services by a user/coordinator of system 1, or independently of the provision of such services, to enter new study participants 26.

A trips view 90, exemplified in FIG. 5, may receive from database 2 information from trip dataset 400 about trips associated with one or more studies and displays it, including for example, trip status (occurred, submitted, in progress, upcoming, booking requested, etc.), travelers and itinerary (e.g., traveler name, dates, responsible user, origin, destination), and other information. Trips view 90 may be configured to only display information from trip dataset 400 associated with travelers that a logged in user is authorized to view, for example if a concierge user accesses trips view 90, GUI 43 will only receive and display trip data 400 for trips associated with participants 26 who are assigned to the concierge user. Another user (e.g., a system administrator, manager, etc.) who is permitted to view all participants in data 10, may be able to view the trips associated with all participants. Trips view 90 interface may provide data filter controls 90a, 90b, 90c to filter the displayed information from trip dataset 400 by, for example, trip status, a clinical site or study, specific trip or participant, and by any other data (e.g., by typing a search term). GUI 43 may be configured to enable a user to utilize control 90c associated with a traveler or trip to switch to another interface, for example, participant interface 52. If trip view 90 is filtered by a participant, it may be displayed as participant trips module 56, and may display a travel control 485 allowing the planning of a new trip for that participant 26.

FIG. 6 illustrates an example of travelers view 91, which may receive (e.g., from database 2) and displays study participant data 300, and may also provide controls 91a, 91b to filter the display of travelers view 91 by a specific traveler/participant (traveler, participant, and patient are used interchangeably for clinical study participants). Travelers view 91 may also provide controls 91c (e.g., “Add Participant” button), allowing a user to enter information about a new traveler, or 91d (e.g., a link, url, associated with a participants) allowing a user to switch to participant interface 52. If travelers view 91 is filtered by a study 25 it may be displayed as study participants interface 50 travelers view 91 and study participants interface 50 may be configured to only display participant dataset 300 that a user currently accessing GUI 43 (e.g., logged in) is assigned to, or authorized to view.

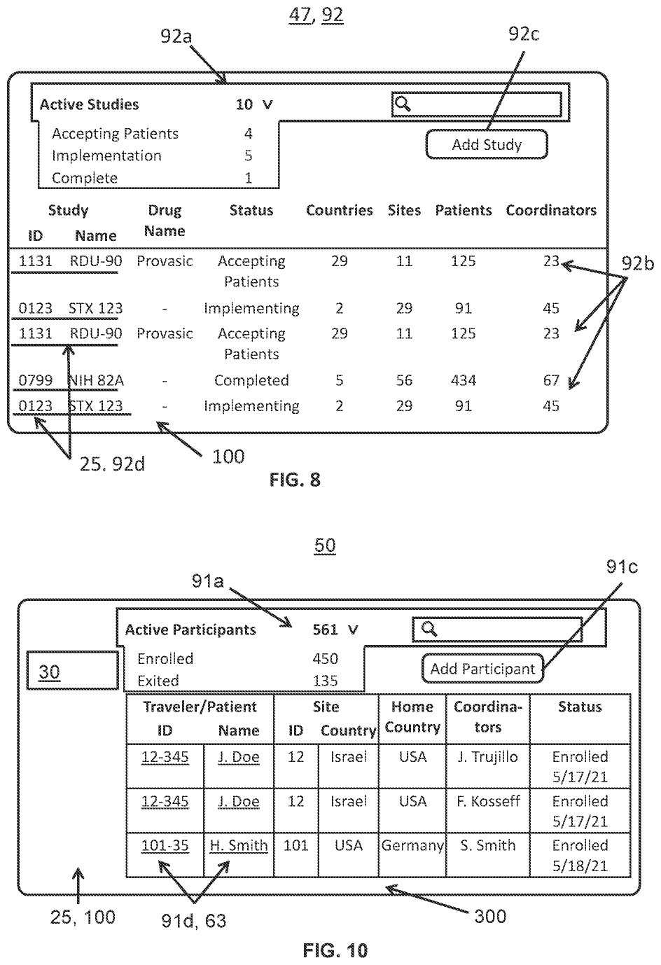

Studies view 92, as exemplified in FIG. 8, receives and displays clinical study dataset 100 data for one or more studies 25, and may provide controls 92a, 92b, 92c, 92d to filter the displayed data by various parameters (e.g., country, type, etc.), add a new study (e.g., “Add Study” button), or switch to the study interface 98 (e.g., utilizing control 92d associated with a study) exemplified in FIG. 9.

Study interface receives clinical study dataset 100 for a clinical study 25 from database 2. Study interface 98 may comprise a study summary area 30 displaying summary information from clinical study dataset 100. Study interface 98 comprises one or more interface modules/panels, having data views, interactive panels, and/or interfaces, related to clinical study 25, including, study overview (not shown), assignments interface (not shown), study participants interface 50, participant interface 52, study sites interface 60, travel policies interface 70, visit schedule interface 80, study coordinators interface (not shown), and others. GUI 43 may be configured to provide navigation controls 31 within study interface 98 allowing a user to utilize the navigation controls 31 to switch to different views or panels of study interface 98, or interface 47, to view, input, or manipulate data about clinical study 25, and manage participants 26.

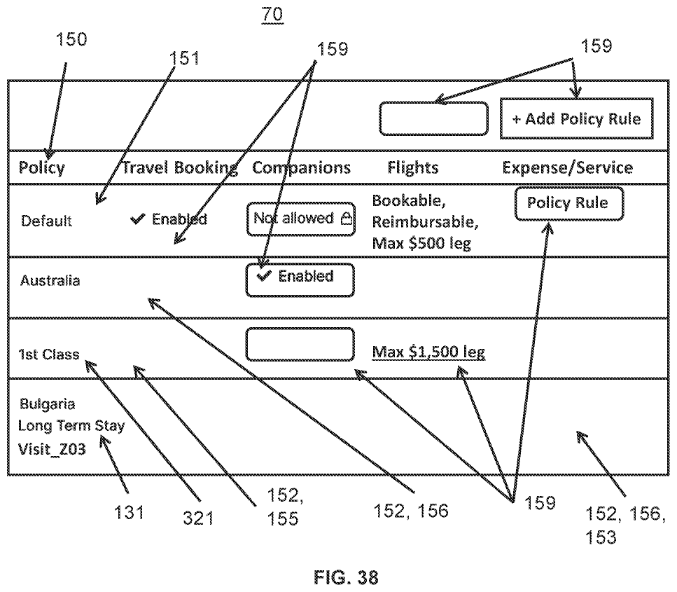

FIG. 38 illustrates an embodiment of a travel policies interface 70 that may receive data about study travel policy 150 for clinical study 25 from database 2, from input device 41, through direct data transmission, and other methods, and may display the study travel policy 150. Travel policies interface 70 may be configured to enable a user to utilize various controls 159 to modify and customize the study travel policy 150 data so that different travel policies apply to different types of travel or travel circumstances. Embodiments of travel policies interface 70 may be configured to require an approval (from, e.g., manager) to create and/or customize a specialized travel policy 152, traveler policy labels 321, and/or visit policy labels 131a. If approval is required, additions of and/or changes to a specialized travel policy 152, a traveler policy label 321, and a visit policy label 131 may occur after approval is granted, which may be granted once or on a per need basis.

For example, travel policy interface 70 may provide a travel rules control 159 that enables a user to add new, import, preview, customize, add to, or modify, a study travel policy 150, including a main study travel policy 151 and/or a specialized travel policy 152 and store them in database 2. Travel rules control 159 may further enable a user to create and store in database 2 a traveler label 321 and a visit policy label 131 associated with a specialized travel policy 152. Control 159 may be utilized to create other types of policy labels associated with specialized travel policies 152, such as country policy labels, site policy labels, site location policy labels, and various other travel endpoints, or circumstances.

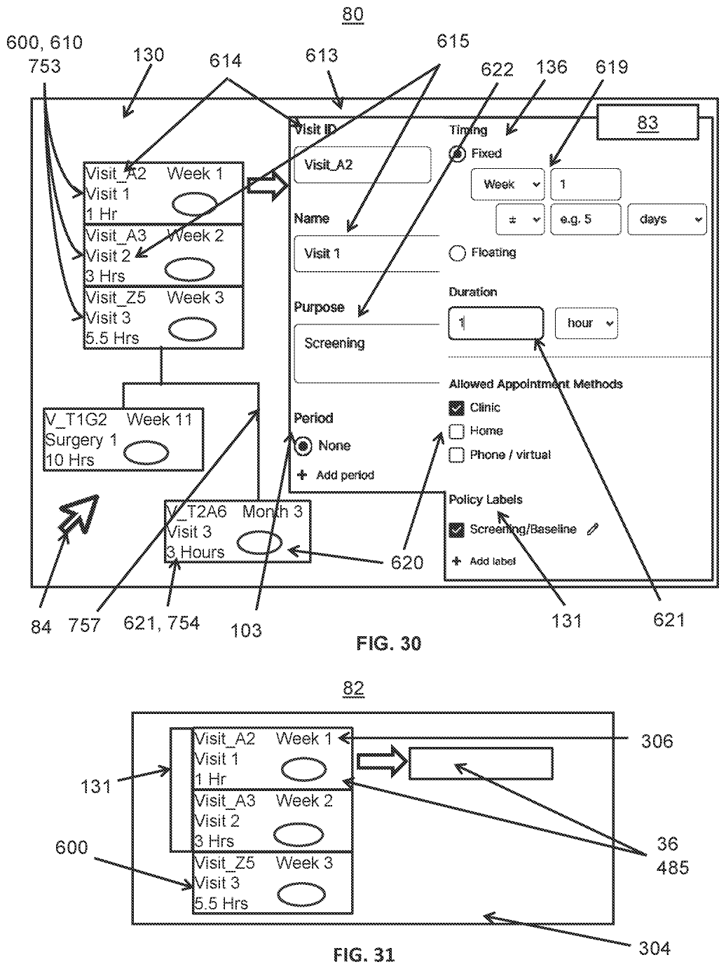

FIG. 30 illustrates an embodiment of a visit schedule interface 80 that receives (e.g., from database 2, input device, data transmission, etc.) and displays study visit schedule 130 data. Visit schedule interface 80 may be configured to enable input, correction, or customization of study visit schedule 130 data utilizing input device 41. Visit schedule interface 80 may provide controls 83 that are configured to modify study visit schedule 130 data by adding, or modifying information about, visits, visit tracks (e.g., clinical protocol arms or patient groups, visually represented as a branch or track in the visit sequence), visit cycles (e.g., repeating one or more visits until a condition is met), visit jumps (e.g., if conditions are met a participant may omit or repeat visits, switch arms/groups, and in general switch to another point in the visit schedule as defined by the configuration of the jump node), information (e.g., about a visit, group of visits, the visit schedule), and others. Visit schedule interface 80 may also provide controls 83a to enable a user to add a visit policy label 131 to study visit schedule 130. Visit policy label 131 may be associated with one or more visits, tracks, cycles, and other visit schedule attributes 613 enabling customizable travel policies.

Participant visit schedule module 82 receives (e.g., from database 2, input device, data transmission, etc.) and displays participant visit data 304 for a participant 26. In a preferred embodiment, visit schedule interface 82 may be configured to provide a participant visit control 36 and participant travel control 485 associated with a current visit 306 from participant visit data 304. A user may utilize control 36 to skip a current visit 306, and control 485 to begin planning a new trip and switch to travel interface 500. Participant visit control 36 may also be associated with other visits from participant visit data 304, in which case a user may utilize control 36 to fast forward to that visit, or to make that visit a current visit 306.

Study visit schedule 130 is a dataset comprising data about a clinical participant clinical study visit, also referred to as an appointment, during which a clinical participant may be examined or assessed as part of a clinical study 25. Data in study visit schedule 130 may comprise a visit id 614, visit name 615, visit number 617, an appointment procedure 135, an appointment schedule 136, visit duration 621 (e.g., associated with the visit name, visit number 617, and/or appointment procedure 135), and exit strategy. Appointment procedure 135 may be associated with, or it may be based on, one or more of the visit id 614, visit name 615, visit number 617, visit tracks, and other visit attributes 613. Appointment schedule 136 may indicate a specific date for a visit, a visit time window 619 with a range of dates (e.g., specific date range; a date range relative to a previous visit, relative to the beginning or end of a study, or relative to another prior or later event or date). Appointment schedule 136 may also indicate a “floating” appointment schedule 136 indicating that the visit (or appointment) can be scheduled at any available time. Appointment schedule 136 may also comprise an appointment start date and time, an appointment end date and time, and/or appointment duration 621. Appointment schedule 136 may also comprise an arrive-by-date-and-time 137 and a depart-after-date-and-time 138. Arrive-by-date-and-time 137 may be based on an appointment start date and time, the appointment procedure 135, and/or other factors, allowing a patient to settle mentally or physically prior to an appointment, to complete pre-appointment preparations, or to accommodate patients with medical or health issues. Depart-after-date-and-time 138 may be based on an appointment end date and time, the appointment procedure 135, and/or other factors, allowing a patient time after an appointment procedure 135 for observation, calming, follow-up, accommodation for health or medical issues, and others.

Study visit schedule 130 may also include information about an unscheduled visit that may be used for an unscheduled medical follow-up that may be discretionary or may be mandated after certain appointment procedures 135. Study visit schedule 130 may also include data about clinical study visit tracks 700 (e.g., patients undergoing different schedule of assessment, or tracks, depending on various criteria), visit cycles (e.g., repeating one or more visits/appointments), visit “jumps” (depending on criteria, a patient may jump, e.g., out of a visit cycle; to a different visit track; to exit a study; to another visit, and others), visit notes, and other visit data attributes 613.

Visit id 614 may be an alphanumeric identifier for a visit in a study visit schedule 130. For example, it may be an abbreviation reflecting one or more of a visit name 615, visit number 617, visit description 622, appointment procedure 135, and other visit data attributes 613. Visit name 615 may be a descriptive name for each visit. Visit name 615 may be the name of a visit from an FDA approved study protocol.

Exit strategy may describe how a patient has exited (e.g., due to an Exit Reason 710), or will exit (e.g., rollover to another study if no early exit), a study, preferably based on personal information 307 of participant 26. For example, a study visit schedule may comprise multiple exit tracks that allow for participants to exit a study based on different exit reasons 710 if participants are in different circumstances. A participant who completes a visit schedule may have an end of study (EOS) visit following which the participant may exit the study. Other patients may exit one study and enter another, such as a long-term extension or open label extension study. A patient may also exit a study for various exit reasons 710 (e.g., by choice, adverse effects, illness, death). An exit strategy may be configured depending on the method of exit (e.g., study completion, rollover, early exit reason 710) to move the patient to one of several exit tracks, which may comprise different visits. In an example, early exit due to adverse event exit reason 710 may require an exit track with numerous safety follow-up visits, whereas a patient who completes the entire visit schedule may just attend an end of study visit.

A study participants interface 50, as illustrated in FIG. 10, may display a list of participants in a study who have opted to use the services facilitated by a system according to the present invention, such as a concierge like travel booking, expense reimbursements, and others. Study participants interface 50 may be configured to display a participant interface 52 by utilizing a control 63 associated with a participant 26 (e.g., clicking on a participant). Participant interface 52 may also be accessed by utilizing the control panel 44, or the main search control 45.

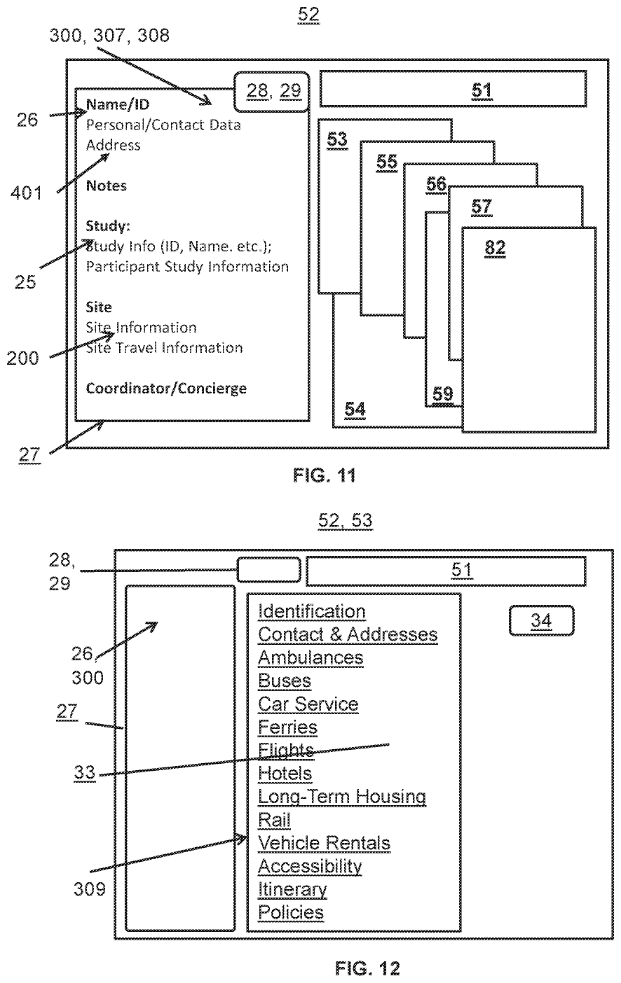

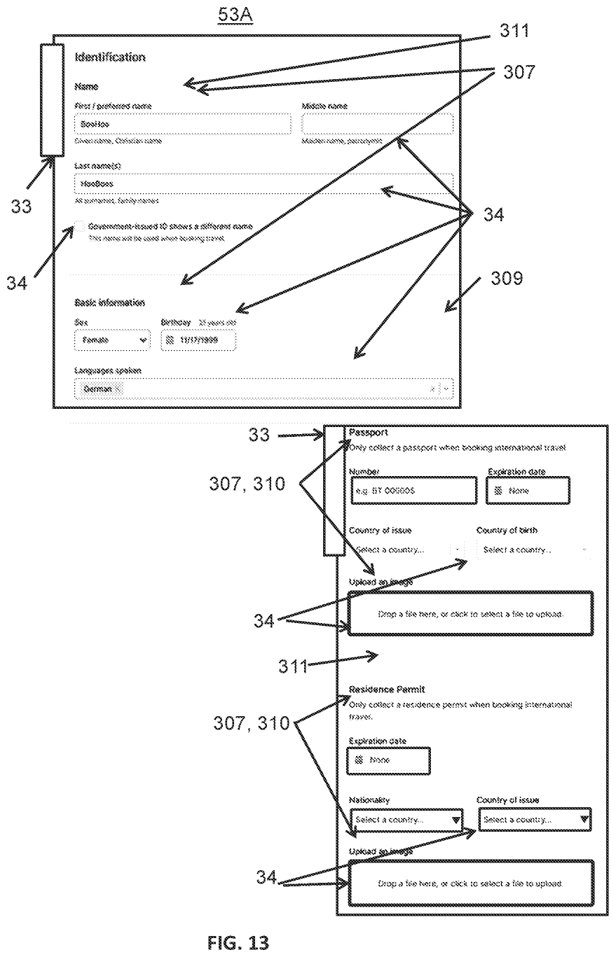

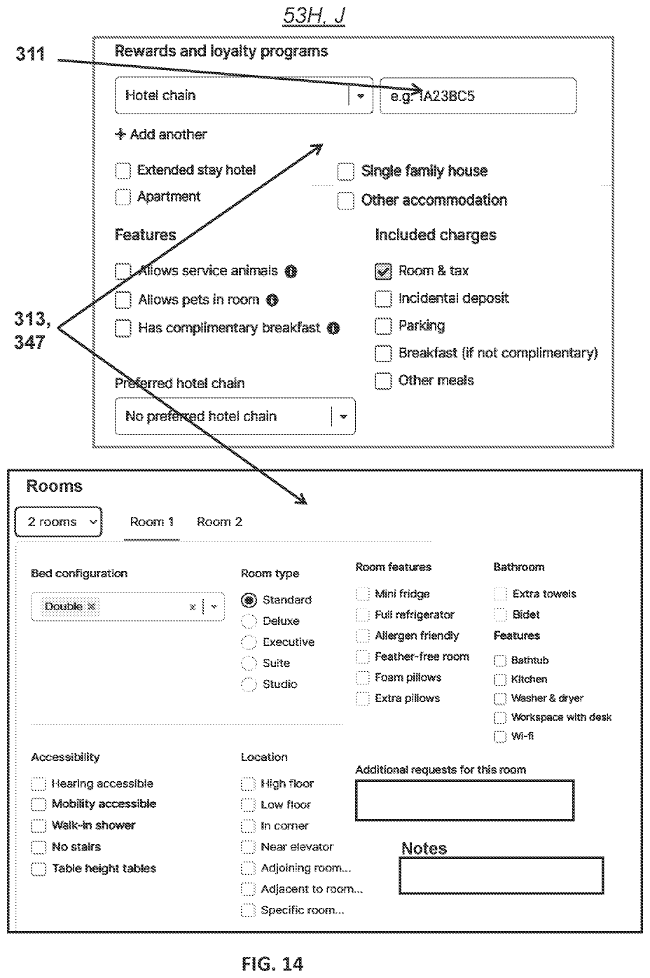

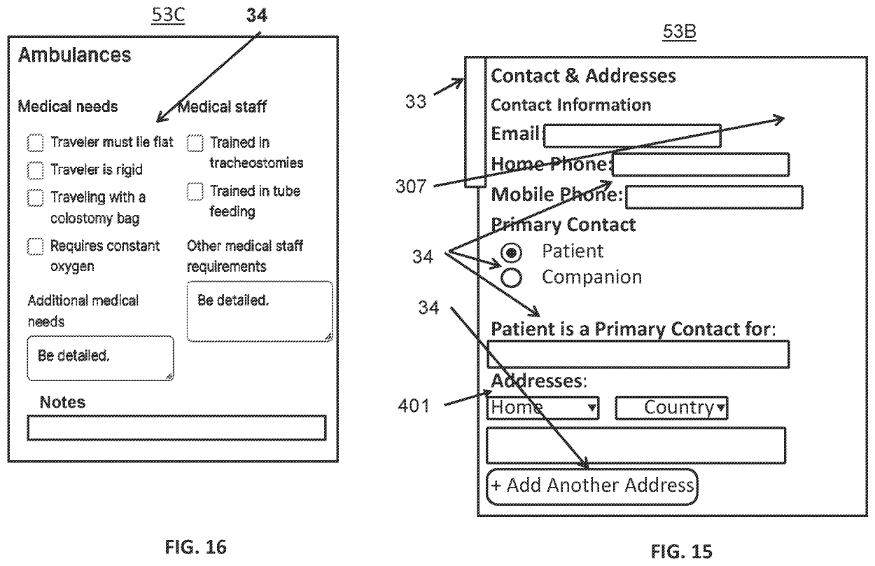

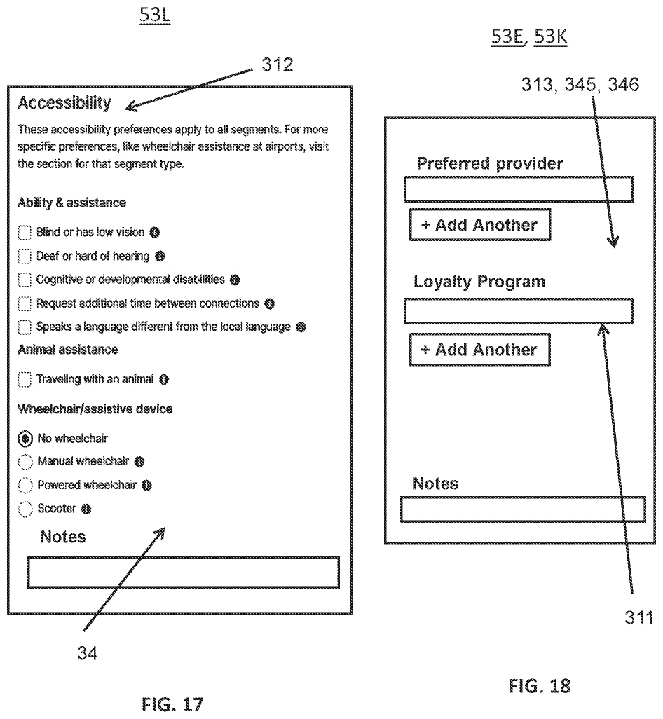

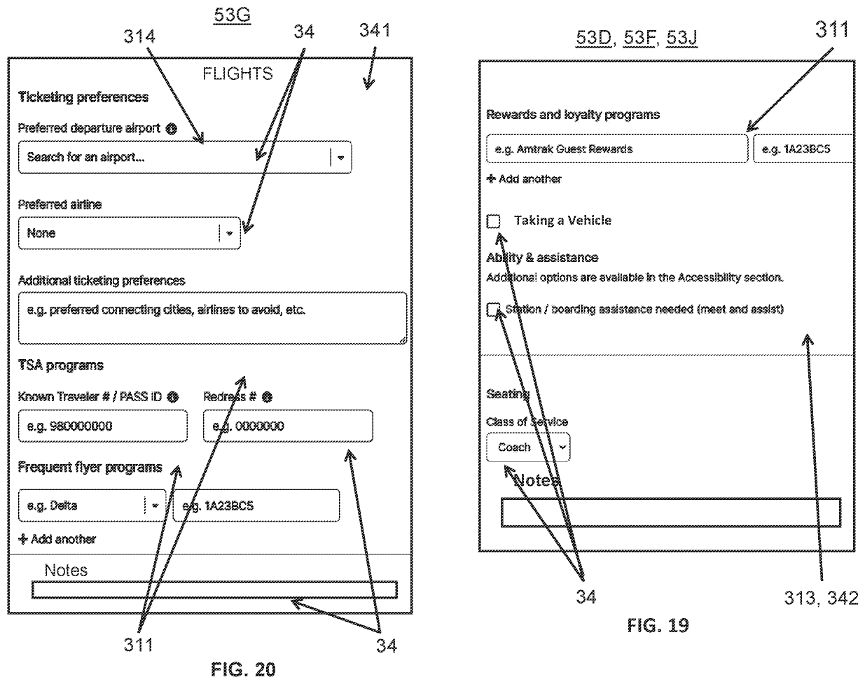

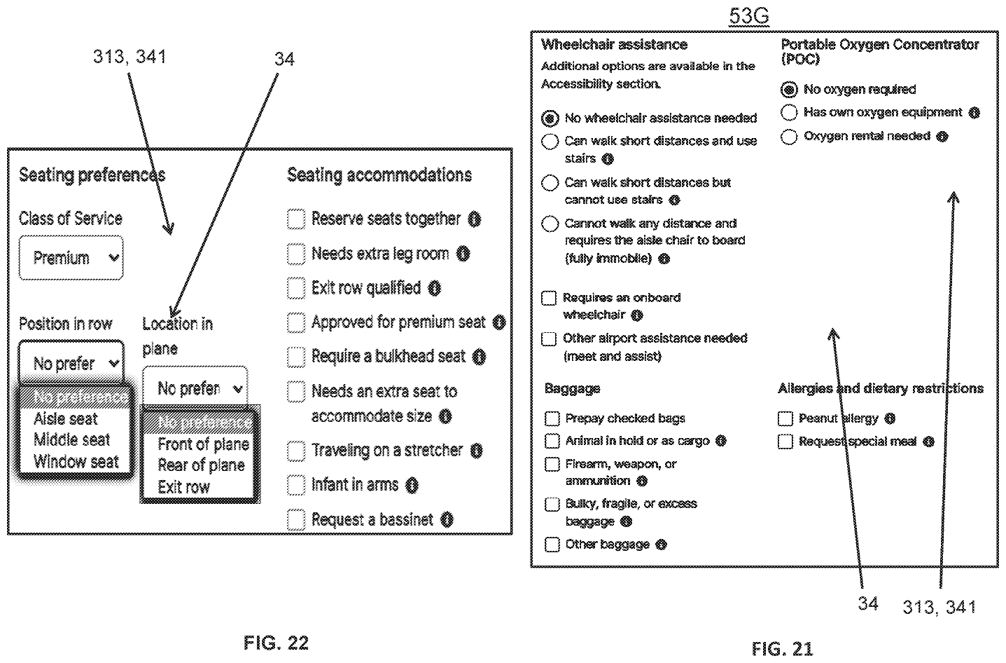

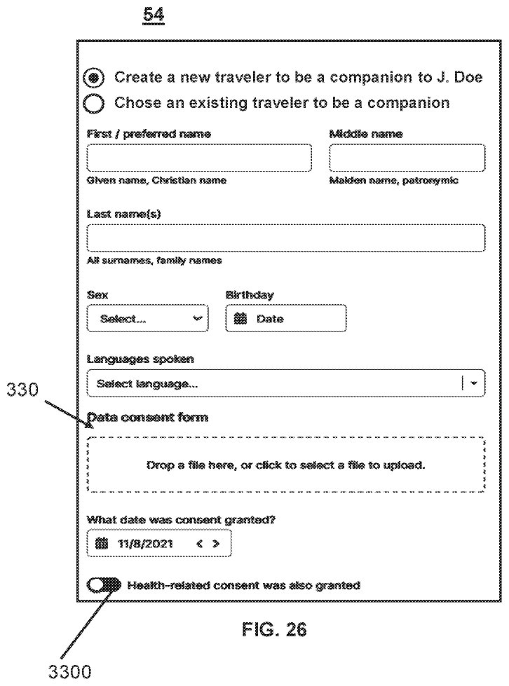

Participant interface 52, exemplified in FIGS. 11, 12, may receive participant dataset 300 about participant 26 from one or more of database 2, input device 41, network data transmission and other methods, and may display participant dataset 300. Participant interface 52 may comprise a summary area 27 displaying information from participant dataset 300. Participant interface 52 may comprise participant data controls 28 configured to enable a user to input, correct, and customize participant dataset 300, by utilizing controls 28 and/or input device 41. For example, by utilizing control 28, a user may enter, or modify participant id, participant site, notes concerning participant 26, participant enrollment date, and may also rollover participant 26 from on clinical study 26 to another clinical study 26 (e.g., an extension study), may exit a participant from a study (including provide an exit reason 710), remove or add a participant as travel companion (exemplified in FIG. 26), and various other functions. Participant interface 52 may also be configured to allow a user to utilize site data controls 29, and coordinator controls (not shown) directly from participant interface 52. Participant interface 52 may comprise one or more interface modules having one or more of interactive data views, panels, and interfaces, including travel profile module 53, companion module 54, participant travel policies module 55, participant trips module 56, participant consent interface 57, participant visit schedule module 82, and participant expenses interface 65, each of which may be displayed by the GUI responsive to a user utilizing a participant control 51.

Travel profile module 53, exemplified in FIGS. 12 26, receives traveler profile 309 data from database 2, and provides controls to input, change, modify or customize traveler profile 309. Travel profile module 53 may be viewed in a identification view 53a, contact & addresses view 53b, ambulances view 53c, buses view 53d, car service view 53e, ferries view 53f, flights view 53g, hotels view 53h, long-term housing view 53i, rail view 53j, vehicle rentals view 53k, accessibility view 53l, itinerary view 53m, policies view 53n, each of which may be configured to display, and enable input and modification of, traveler profile data 309. Travel profile module 53 may comprise traveler profile navigation control 33 allowing a user to utilize it to switch one of the travel profile views 53a-53n, and a traveler profile control 34 allowing a user to utilize it to input, modify, or manipulate traveler profile data 309. Traveler profile navigation control 33 may also be configured to allow a user to utilize it to switch any other interface 47, or to any view, module, or interface within participant interface 52, including companion module 54, participant travel policies module 55, participant trips module 56, participant consent interface 57, participant visit schedule module 82, and participant expenses interface 65.

Travel profile module 53 may also be configured to transmit traveler profile 309 to be stored in database 2. Companion module 54, receives companion dataset 325 from database 2, and provides controls to input, change, modify or customize companion dataset 325. Companion module 54 may also be configured to transmit companion data to be stored in database 2.

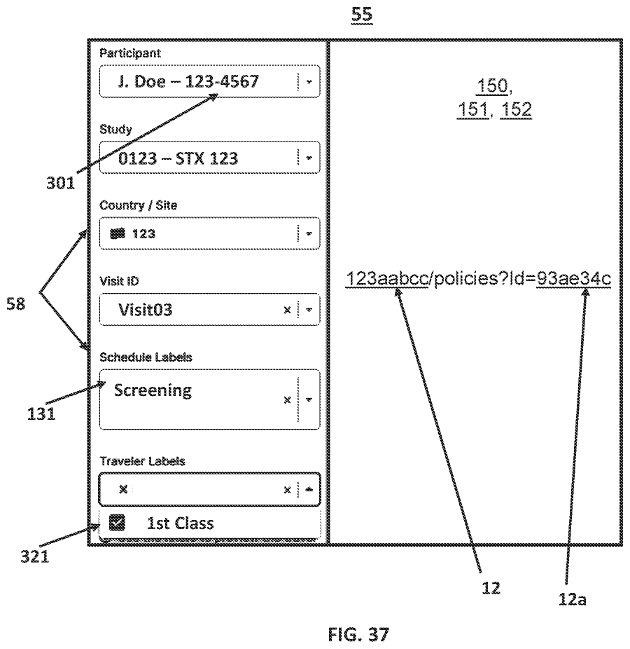

Participant travel policies module 55, exemplified in FIG. 37, enables viewing and customization of participant data 300 related to study travel policy 150 applicable to a participant 26 for a clinical site visit trip, based on main travel policy 151 and any applicable specialized travel policies 152. Participant travel policies module 55 may be configured to display study travel policies 150, including the main study travel policy 151, and any specialized travel policies 152 associated with participant 26 and applicable to participant 26 trips. Participant travel policies module 55 may be configured to allow a participant to be associated or dissociated with a specialized travel policy 152 by utilizing controls 58 to add or remove an association of participant 26 with a traveler policy label 321 and/or a visit policy label 131. Embodiments may be configured to require an approval (from, e.g., manager) to create and/or customize a specialized travel policy 152, traveler policy labels 321, and/or visit policy labels 131a. If such approval is required, specialized travel policy 152, traveler policy labels 321, and visit policy labels 131 additions and changes may occur after approval is granted.

Participant trips module 56 may display information about past and upcoming participant trips, based on data from a trip dataset 400 and a participant dataset 300 received from database 2. Participant trips module 56 may display information identifying a participant who is taking the trip, a trip status (e.g., pending, submitted, requested, booked, confirmed, etc.), a trip destination, method of transportation, trip schedule, and other information related to a participant trip. Participant trips module 56 may be configured to provide travel control 485 that enables a user to switch to the travel planning interface 500 (e.g., to plan a new trip, modify existing one), or clickable link control 90b embedded in an individual trip text enabling a user to display an individual trip 59 module.

Individual trip 59 module may be configured to receive from database 2, and display, itinerary 475 data, and also may be configured to enable modification of itinerary 475, for example trip details 476, trip planning history 477, trip segment data 501, itinerary message 478, and other information. Individual trip 59 module may also provide controls enabling a user to switch to other interface modules of GUI 43 and to enter and/or modify various data. For example, a user may utilize control 480 to cancel a trip, or to request booking of trip segments by transmitting to booking module 575 data necessary for travel booking, such as booking request data 451. Control 480 may also be utilized to send itinerary 475 to one or more recipients according to traveler contact information 308 and/or the site contact information 202, by utilizing transmission methods and transmission destinations, or recipients, provided in traveler contact information 308 and/or the site contact information 202. For example, itinerary 475 may be sent by emailing it to an email address provided in traveler contact information 308 and/or site contact information 202, or may be sent by facsimile if so indicated in traveler contact information 308 and/or site contact information 202. Traveler contact information 308 and/or site contact information 202 may also indicate that itinerary 475 and other information may be sent by transmitting over a network connection (using, e.g., API calls, secure data/file transfer protocols, and other methods) to a computing system capable of receiving itinerary 475. Such a computing system may be a system associated with a clinical site system, a hospital or another facility, with a patient (e.g., mobile device), and others. Embodiments of the invention may be configured to enable secure online viewing and/or downloading of itinerary 475 or other information by providing a secured portal accessible by authorized persons, for example a contact in a traveler contact information 308 and/or in a site contact information 202, who may be notified (according to the contact information) to log in to the portal. For privacy and security reasons, control 480 may be configured to only allow itinerary 475 to be sent to pre-authorized recipients provided in traveler contact information 308 and site contact information 202.

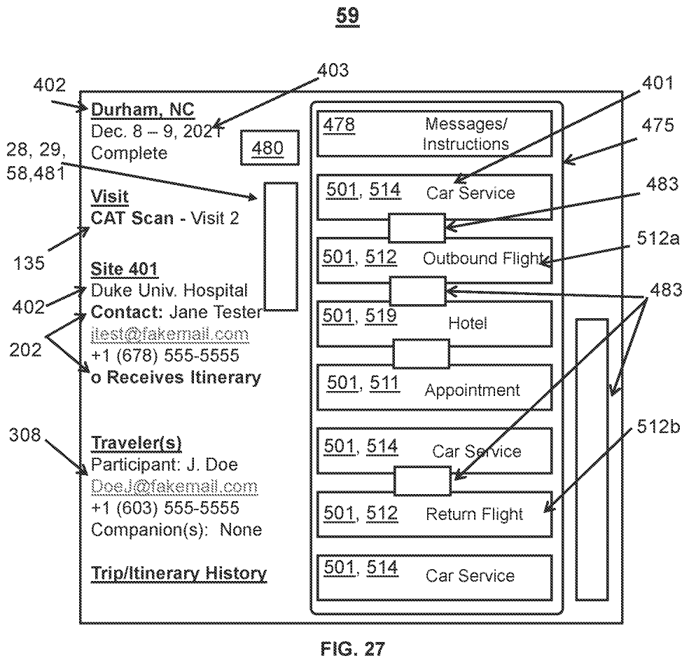

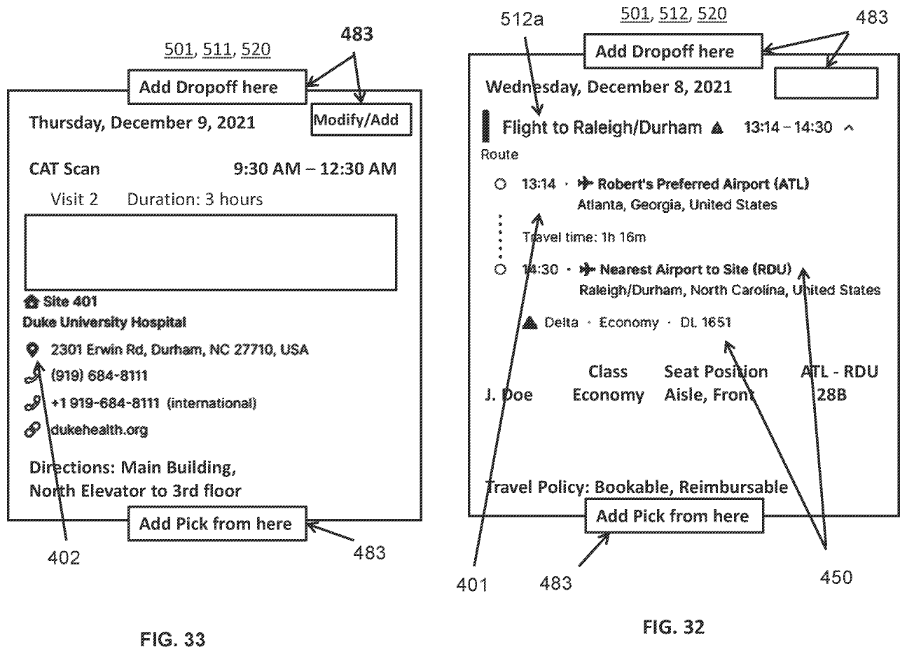

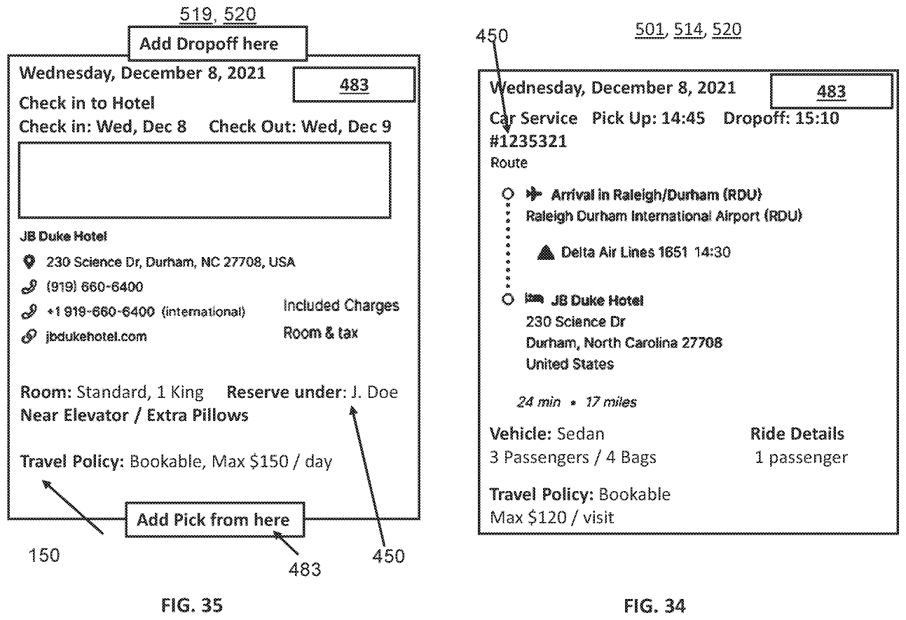

An individual trip 59, exemplified in FIG. 27, module may also be configured to provide controls 481 enabling a user to modify a traveler policy label 321 associated with participant 26 or to add/remove travelers (such as travel companions) to itinerary 475. Individual trip 59 module may also be configured to provide controls 483 to add or modify trip segments by causing the GUI to load the travel planning interface 500, or trip segment panels 520 as exemplified in FIGS. 32-36. For example, control 483 may be associated with a first trip segment start location 503 or with a first trip segment end location 504 of a first (e.g., existing) trip segment data 501, so that a user may utilize control 483 to add a second (e.g., new) trip segment 501 having a second segment start location 503 or a second segment end location 504 based, respectively, on the first trip segment end location 504 or the first trip segment start location 503 of the first (e.g., existing) trip segment data 501. For example, if control 483 is associated with a first trip segment start location 503 at a departure airport, a user may utilize control 483 to add a second car service trip segment 514 having a second trip segment end location 504 be the departure airport location.

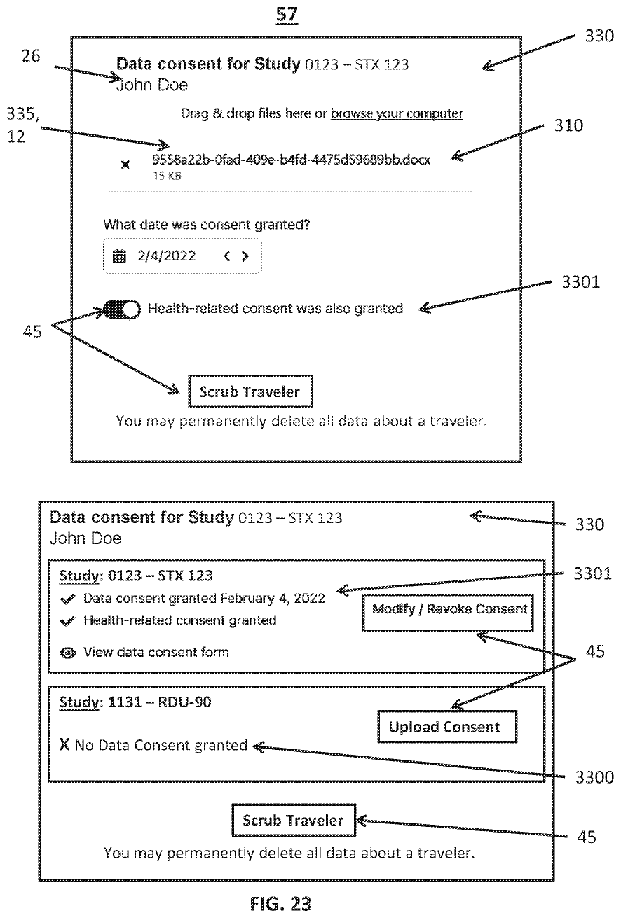

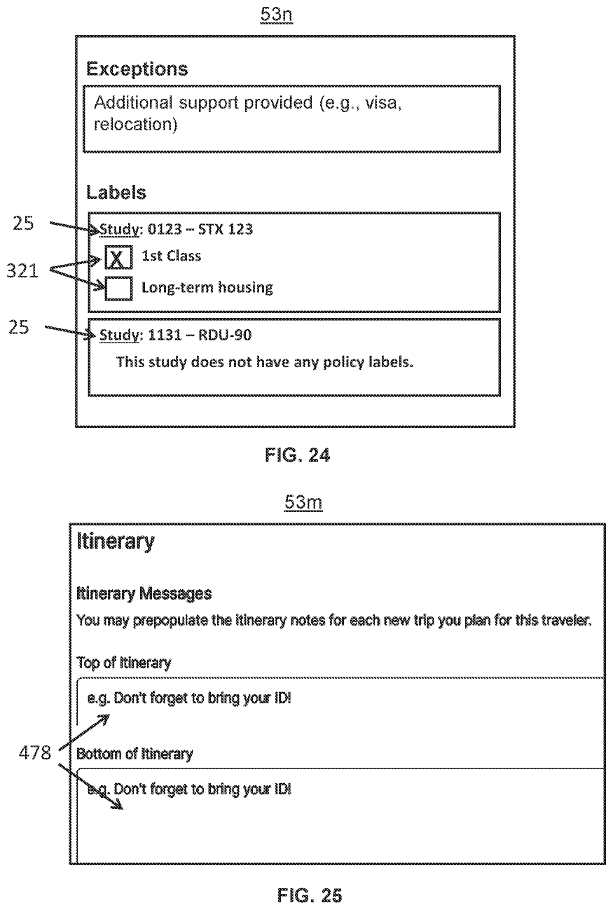

FIG. 23 illustrates an embodiment of a participant consent interface 57 receives from the database 2, and displays participant consent record 330, and may also provide consent controls 45 (e.g., slider, checkbox, button, upload controls) configured to allow modification of participant consent record 330. For example, a user may utilize a consent control 45 to indicate whether participant 26 has provided informed consent for the use of personal information, including health information. Consent control 45 may also be used to upload signed personal information data consent forms 335, for personal information and/or health records. Consent control 45 may also be configured to initiate a scrubbing (e.g., indicated as “scrub traveler”), or removal, of personal information and health records data of a participant 26, or of all data of a participant 26, from system 1.

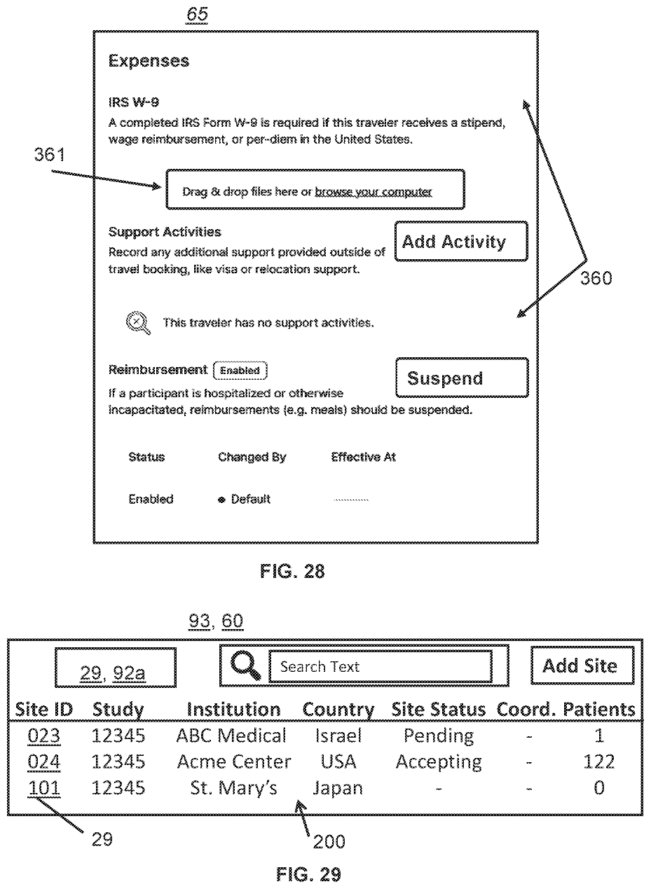

Participant expenses interface 65, exemplified in FIG. 28, receives from the database 2, and displays participant expense data 360, and may also provide expense controls allowing a user to modify and change participant expense data 360. For example, a user may utilize the expense controls to receive (e.g., import for a computer or over a network, drag and drop, etc.) participant tax data 361 (e.g., Tax ID, SSN; IRS forms), indicate support activities provided to a participant 26, add or change status of expenses, and expense rules and policies (e.g., whether reimbursable, expense limits). GUI 43 may also be configured to enable check or validation of tax information (may be initiated automatically or by a user) received for example, from Participant Expenses Interface 65, a W9 form, or elsewhere. In some embodiments check or validation of tax information may involve utilizing the US Internal Revenue Service (IRS) Taxpayer Identification Number (TIN) Matching Tools, by for example, providing, transmitting, and/or querying tax information to an IRS TIN Matching Tool and receiving from the IRS TIN Matching Tool information about the tax information, for example, TIN validation information.

Sites view 93 receives from database 2, and displays clinical site datasets 200 for clinical sites associated with one or more clinical studies 25. Sites view 93 may provide controls to filter the displayed data by various parameters (e.g., by study, country, search keywords, and others), for example by study 25, and display site view 93 as study sites interface 60. Site controls may also be utilized to switch to study sites interface 60 or site interface 99, and to enable other functionality (e.g., “add site”). Study sites interface 60 receives and displays clinical site dataset 200 for sites associated with a clinical study 25. Study sites interface 60 is configured to enable a user to input, correct, or customize clinical site dataset 200 by utilizing a site control 29 and/or input device 41.

Site interface 99 may comprise one or more data views, interactive panels and/or interfaces, including, site participants module (e.g., study participants associated with a site), site assignments module (e.g., users, concierge users, and other persons assigned to study participants associated with a site), site travel policies module (e.g., travel policies associated with a site, including site labels, visit labels for visits at the site, and traveler labels for participants associated with the site), and others. The site interface may provide a site control 29 allowing the addition of a new clinical site, and modification of clinical site data 200.

Coordinators view 94 receives and displays data about users/coordinators associated with one or more studies, and may provide coordinators view controls to filter the displayed data by various parameters (e.g., by study, country, site, coordinator, participant, etc.), to add user/coordinator, and to switch to study coordinator interface.

Private cloud 6 may be any combination of networked computing resources (e.g., databases, servers, gateways, etc.) that are securely isolated from the internet (using, e.g., private networks, firewalls, secured gateways, etc.).

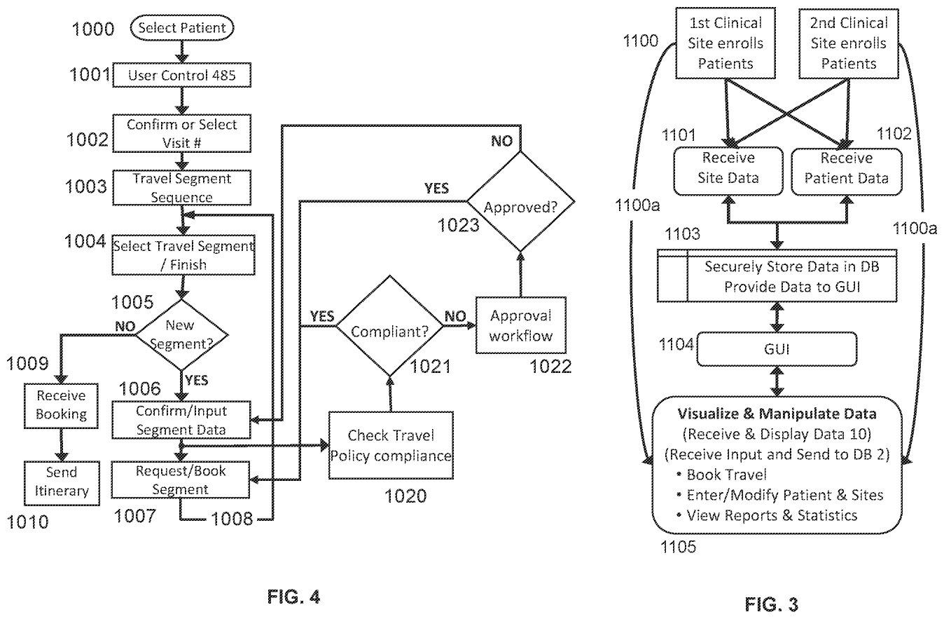

Encryption tools 11 may be configured to encrypt data 10 at rest, while stored in database 2, and to unencrypt data records of data 10 when those data records are accessed by GUI 43, or by another authorized computing resource. Encryption tools 11 may be configured to encrypt the entire database 2 (using, e.g., a transparent data encryption (TDE)), or specific records, cells, or data in Database 2. Encryption tools 11 may be configured to encrypt data being sent to Database 2 (e.g., client side encryption), for example in steps 1101, 1102, 1104 of FIG. 3, and/or upon storing data in Database 2 (e.g., server side encryption), for example in Step 1103.

Internet gateway 7 may be utilized for enabling secure communications between the private cloud 6 and the internet, and enable secure transmission of personal information. In some embodiments private cloud 6 may comprise a public subnet (e.g., publicly accessible from the internet through internet gateway 7) and a private subnet isolated from internet gateway 7. In a preferred embodiment, private cloud 6, server 3, internet gateway 7, and workstation 40 communicate using https protocol utilizing transport layer security (TLS) encryption of all data transmissions between database 2 and workstation 40. For added privacy protection and data security workstation 40, internet gateway 7, server 3, and other devices within system 1, may be configured to prevent caching personal information to non-volatile storage (e.g., disk, ROM) and instead use only volatile computer memory (e.g., RAM) to transmit/display personal information.

Monitoring tools 20 may be utilized to monitor, log, and enable auditing of, access and communications between the internet, workstation 40, server 3, private cloud 6, and/or database 2. Monitoring tools 20 are known in the industry and may include cloud monitoring software or services (e.g., CloudWatch, MetricFire, Datadog, Dynatrace, Prometheus, Graphite, and others.), and logging tools (e.g., AWS VPC Flow Logs) that log and may provide audit trails for connections to private cloud 6 that attempt to access, process, transmit, and/or store data 10, including Personal Information.

To implement granular access to private cloud 6, server 3, and database 2, system 1 may utilize an identity and security management (IAM) service or system 21. IAM 21 may identify, authenticate, and control access for individuals as well as hardware and applications that may need access to database 2, servers 3, or specific datasets or subsets, such as participant dataset 300 and allows users to only access data the users are authorized to access. For example, when a concierge user is performing tasks through the GUI 43 (e.g., booking participant travel), an embodiment of the present invention may utilize IAM 21 to restrict GUI 43 from accessing and displaying Participant dataset 300 for participants who the user is not authorized to view. In a preferred embodiment, IAM System 21 is configured to restrict access to all Data 10 by user, and by user access level (e.g., user role) so that each user is allowed to access only the portion of Data 10 that the user is assigned or authorized to access. Access to server 3 also may be restricted by user, and user access level. User authentication may be handled internally, or by various existing platforms (e.g., Okta, OneLogin, Auth0, RSA SecurID, SecureAuth Oracle Access Management Suite). Embodiments of the present invention may also utilize multiple factor authentication in addition to username and password to increase data security.

Data 10 in database 2 may comprise one or more clinical study datasets 100 containing data subsets and information related to a plurality of clinical studies. The terms dataset and data subset generally are used interchangeably here, and should be understood broadly to include any set, collection, record or aggregation of data or information, in any form, created, received, or provided in relation to a clinical study 25 and can be of any size (including a single datum), and in any form, including, numbers, text, audio, video, images, documents, spreadsheets, and others.

Clinical study dataset 100 may comprise a plurality of data subsets (or sub datasets) about a clinical study 25, including protocol data 101, user/coordinator data 115, study visit schedule 130 dataset, clinical site dataset 200, participant dataset 300, and trip dataset 400. User/coordinator data 115 comprises information about users who are authorized to access the travel booking system, as well as users' assignments to one or more travelers. Protocol data 101, for example illustrated in FIG. 9, may comprise information that generally may be applicable to a study as a whole (e.g., the study protocol), and may include the study sponsor, study id, study name, study status, duration, start and end dates, drug or treatment information, study phase, medical or scientific area, appointment procedures 135 (e.g., drug or treatment administration, consultation, medical examination, MRI, CAT scan, PET scan, lab work, infusion, LP, surgery, PK sample collection, radiology, ultrasound), types of participants (e.g., gender, age, medical condition, environmental exposure, nationality, region, and others), and other relevant information.

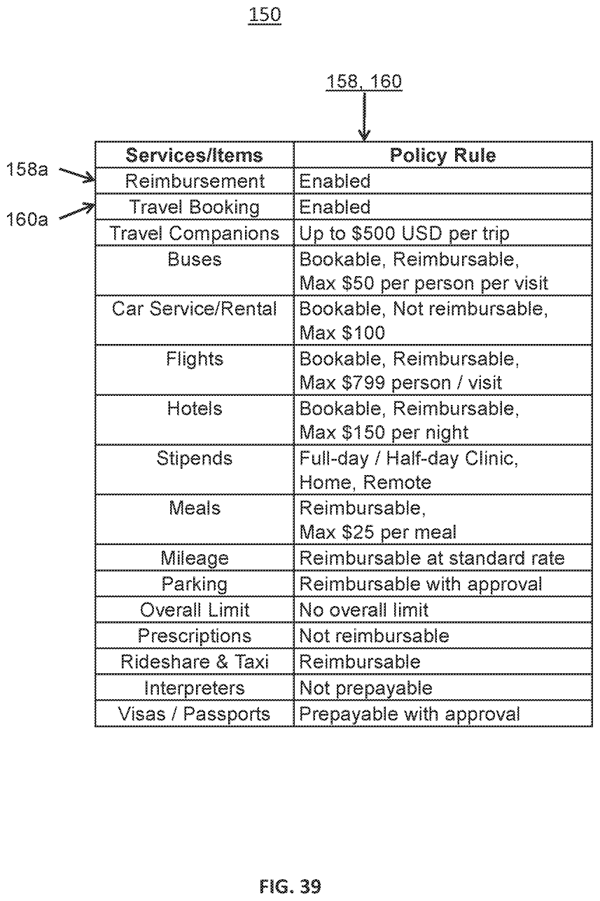

Study travel policy 150 may comprise data about rules, options, restrictions, and policies applicable to travel by participants in a clinical study 25. For example, study travel policy 150 may comprise reimbursement information (expense rules) 158, such as, whether various travel related expenses (e.g., flight, cars, hotels, transportation, meals, tolls, parking, prescriptions, phone calls, and others) are reimbursable, maximum reimbursable cost, and whether approval is required. Study travel policy 150 may also comprise travel rules 160 indicating whether certain travel services are bookable through system 1, and their maximum costs, such as air travel, car rental, car service, ferries, ambulances, rail, buses, hotels, long-term lodging, and others. FIG. 39 illustrates an example of a study travel policy 150 comprising expense rules 158 and travel rules 160. Expense rules 158 may comprise an expense indicator 158a of whether reimbursements are enabled, and travel rules 160 may comprise a travel booking indicator 160a of whether travel services booking is enabled.

Study travel policy 150 may also comprise a main travel policy 151 and a specialized travel policy 152. Main travel policy 151 may apply by default to travel of clinical study participants 26, unless a specialized travel policy 152 applies to one or more aspects of the travel.

A specialized travel policy 152 may establish rules and restriction different from the main travel policy 151, for example, different reimbursement amounts for some travel related services, different allowable cost for certain types of transportation or lodging (e.g., higher cost for flights, car services, higher or lower ranked hotels with different cost limits, permit long-term lodging), may allow booking of certain transportation that may not be allowed under main travel policy 152 (e.g., allow booking of car service, ambulance, or first class airfare that otherwise may not be allowed). For example, a specialized travel policy 152 may be a visit travel policy 153, site travel policy 154, custom travel policy 155, a country travel policy 156, and combinations thereof.

A country travel policy 156 may apply based on country (e.g., origin, destination). A site travel policy 154 associated with a particular clinical site may apply for participant travel to and from the clinical site. A visit travel policy 153 may be based on (e.g., associated with) a visit policy label 131, and/or one or more visits 610 (a visit or visits having, e.g., a visit id 614, a visit type, a visit name 615, visit descriptions 622, and others). A visit policy label 131 may be associated with one or more visits or appointments (e.g., visit 1, visit “sg3,” “surgery” visits, visits 1-8, visit during a clinical period, etc.)

A custom travel policy 155 may be based on a traveler policy label 321 associated with one or more participants 26. For example, a traveler policy label 321 may be a “1st class” label or a “long-term housing,” and a custom travel policy 155 associated traveler policy label 321 may allow booking of first class air travel or long-term housing, even if main travel policy 151 does not allow them. A specialized travel policy 152 may also be based on various other factors (e.g., appointment location, institution, visit city or town, participant age, and others), as well as additional policy labels that may be associated with a participant, trip, site, country, institution, or other circumstances.

A specialized travel policy 152 may combine one or more of a visit travel policy 153, site travel policy 154, custom travel policy 155, and a country travel policy 156. For example, a specialized travel policy 152 combining a “long-term stay” visit policy label 131, a visit travel policy 153 for “surgery” visits, and a USA based country travel policy 156, will permit long-term hotel lodging in the USA for participants traveling for a “Surgery” visit associated with the “Long-term Stay” label 131, even if the main travel policy 150 does not allow long-term hotels.

Clinical site dataset 200 comprises information about a clinical site associated with a clinical study 25 where appointment procedures 135 are performed during clinical site visits to assess, evaluate, and/or examine participants 26. Alternatively, an appointment procedure 135 may be performed at a patient's home (or other non-site location) and the clinical site will perform the assessment upon receiving data from the appointment procedure 135. Certain appointment procedures may also be performed remotely, or virtually, using a mobile device, a portable analytical device, and similar equipment. Clinical site personnel may enroll participants in a study. A clinical site dataset 200 may comprise site appointment locations 201 information about one or more site locations, such as a site main location, and a site appointment location 201. A site appointment location 201 may comprise information about an appointment procedure 135, location address, (identified by, e.g., street, building, office or laboratory number), requirements or directions about participant 26 arrival (e.g., arrive at a different building, at a triage station, or wait for pick up at a different location). A site appointment location 201 may be the same or different from the clinical site's main location. A clinical site may perform all appointment procedures at a single site appointment location 201, or different appointment procedures may be performed at different site appointment locations 201. For example, a first site appointment location 201 may be associated with a first appointment procedure 135 (e.g., MRI, Cat Scans), and a second site appointment location 201 may be associated with a second appointment procedure 135 (e.g., medical observation, labs). Clinical site dataset 200 may comprise a site identifier, site name, and site contact information 202. Site contact information 202 contains information about who to contact (e.g., names, titles, departments, automated systems, etc.), whether the contact is to receive an itinerary 475 and how to provide it, and methods of contacting the clinical site (by, e.g., telephone, email, facsimile, online access portal, API interface, etc.).

Clinical site dataset 200 may be provided by a clinical site and received by and stored into database 2 through the study sites interface 60 of GUI 43, as illustrated by numeral 1100a in the embodiment of FIG. 3. Clinical site dataset 200 may also be imported in the form of a data file (e.g., CSV, etc.), or it may be transmitted electronically over a network using secure transfer protocols (e.g., FTPS, UDP, etc.) exemplified by numeral 1101.

Participant dataset 300, comprises data about a participant 26 in a clinical study 25, including participant study information 301, participant personal data 307, traveler profile 309 data, and other data.

Participant dataset 300 may be received in database 2 using various techniques and methods, an example of which is illustrated in FIG. 3. In step 1100 clinical sites may enroll patients 26 into a clinical study 25. Embodiments of system 1 may receive participant dataset 300 from the clinical sites either directly (if transmitted electronically) as indicated in step 1102, or via input device 41, if the participant data set is provided in hardcopy or as an electronic file as illustrated in steps 1100a and 1105. In step 1105, a user may input participant dataset 300 through participant interface 52 of GUI 43 utilizing input device 41. In step 1104 GUI 43 may transmit participant dataset 300 to database 2 where it is securely stored in step 1103. Participant dataset 300 may be transmitted electronically (using, e.g., API, FTP, UDP, secure file transfer protocols, etc.) from the clinical site or another entity as illustrated by step 1102, and then stored in database 2 in step 1103. A user may add also additional participant data 300 through GUI 43, in a participant “onboarding” process by obtaining and inputting additional participant information to supplement, customize, or individualize various portions of participant dataset 300. The onboarding process may include phone calls, electronic assistance request forms, and other methods to obtain information from participants or other relevant sources. A request to access a secure electronic assistance request form may be transmitted to clinical site personnel, participants, caretakers, and other parties with relevant information and will allow them to securely input the information in participant dataset 300.

For the privacy and security of the participant dataset 300, it may be stored encrypted in database 2 identifiable by a unique identifier 12. Unique identifiers 12 may be associated with the separate data records of participant dataset 300 and utilized to pseudonymize participant dataset 300 and enhance security of personal information. For example, participant dataset 300, or any portion of it may only be accessed by utilizing one or more unique identifiers 12, instead actual information about participant 26. FIG. 2 illustrates an example in which GUI 43 requests (arrow labeled as “request”) from database 2 travel profile dataset 309 for a participant 26 with a unique identifier “1234aabbcc.” GUI 43 will receive (arrow labeled as “receive”) from database 2 a record containing travel profile 309 corresponding to the requested unique identifier 12.

Participant study information 301 comprises participant study id 302 that references a participant 26 in a clinical study 25 without revealing participant name or other personal information. Participant study id 302 preferably is a unique code associated with a participant, and may consist of a clinical site id of participant assigned site 303, and an additional alphanumeric sequence. Participant id 302 may be displayed in GUI 43 or provided in various documentation and may be utilized by parties associated with a study to view study information without revealing a participant's name or other personal information. If a participant invokes a right to forget personal information (e.g., privacy laws), participant id 302 may be used to identify and purge the appropriate personal information. Participant study information 301 may also comprise an enrollment date, participant consent record 330, participant assigned site 303, participant visit data 304, participant country 305, and a traveler policy label 321. Participant id 301 may be associated with a unique identifier 12 corresponding to participant 26, with a participant study id 302, or with a study enrollment id 12a. As an example, FIG. 37 illustrates GUI 43 receiving from database 2 a study travel policy 150 associated with a participant 26 whose participant id 301 is 123-4576, unique identifier 12 is “123aabcc”, and enrollment id 12a is “93ae34c.”.

Participant consent record 330 may contain information about a participant's informed consent related to access, use and/or processing of personal information related to a clinical study 25, for example for travel booking, medical assessments, and other purposes. Participant consent record 330 may comprise data indicators (e.g., in the form of binary flags, such as “0” or “1”, “true” or “false”) that a participant has provided informed data consent in the form of a consent provided indicator 3301, or that a participant has withheld, or not yet provided, informed data consent in the form of consent not provided indicator 3300. Consent provided indicator 3301 and consent not provided indicator 3300 may be distinct indicators or may be a single binary (on/off, true/false) indicator, so that on or true indicates consent provided, and off/false indicates consent not provided. Participant consent record 330 is required for all participants (including their companions or caretakers whose personal information may be needed). Participants must sign a consent to process personal data (including health data), which may vary depending on the applicable jurisdiction (e.g., US, GDPR and non-GDPR, Australia, Russia, Turkey, Canada, and others).

Participant consent record 330, and consent provided indicator 3301 or consent not provided indicator 3300 may be more granular and further comprise indicators of whether participant has provided consent regarding some “special” types of personal information data but not others, such as personally identifiable information (PII), personal data (e.g., PD under GDPR), protected health information (e.g., PHI under HIPAA, “Data concerning health” under the GDPR), and whether an electronic copy of an appropriate signed consent form is included in record 330. In a preferred embodiment an appropriate participant consent record 330, evidencing participant's 26 consent for both personal information and for health records, as well as images of the executed consent forms are required for a consent provided indicator 3301 to be present. Embodiments may require a consent provided indicator 3301 to enable the creation and storage of participant 26 data, including participant personal data 307, traveler profile 309, and others, and may be configured to prevent the creation or entry of a participant dataset 300, or to initiate travel booking, for any participant or companion without a consent provided indicator 3301. Embodiments of the present invention may generate an error message, create notifications, and/or prevent storing of any portion of a participant dataset 300 that does not contain data in the participant consent record 330.

Consent provided indicator 3301 and/or consent not provided indicator 3300 may be set automatically by the system based on the presence or absence of signed consent forms and other factors or may be manually set by a user. In an embodiment, a participant (or companion) may view and e sign disclosure and consent forms for the processing of their personal information where such forms may be provided electronically in a similar manner as the assistance request form described above.

Participant assigned site 303 comprises information about a clinical site associated with a participant in a clinical study 25. A participant may have more than one participant assigned site 303 so that the participant may visit different sites and different times during a study.

Participant visit data 304 represent the study visit schedule 130 association with an individual participant (e.g., study tracks/arms and/or visits to which a participant is assigned) and the participant's progress (e.g., to what stage or visit number a participant has progressed; the participant's current or last visit) within the study visit schedule 130. Participant visit data 304 comprise the visit schedule applicable to a participant based on study visit schedule 130 data, including information about at least one appointment procedure 135 and its associated appointment schedule 136 (e.g., start date/time, end date/time, duration 621, and other attributes). Participant visit data 304 may also comprise information about visits that have already occurred, and a current visit 306 (e.g., visit name 615, and/or number of the next visit that needs to be scheduled). Participant visit data 304 may also associate a participant's trip with a specialized travel policy 152 based on a visit policy label 131 in study visit schedule 130. For example, if a participant is traveling for a visit (e.g., an appointment) with a visit id 614 or visit name 615 associated with a visit policy label 131 in study visit schedule 130, portions of participant's trip (e.g., trip segments) may be subject to a specialized travel policy 152 associated with the visit policy label 131 if applicable. Participant visit data 304 may also comprise a traveler policy label 321 associated with a custom travel policy 155.

Participant personal data 307 comprise identification and other information about a participant, such as name, sex, gender, birthdate, age, personal details, languages, government issued ids, known traveler numbers, and other information. Participant personal data 307 may include participant documents 310 in the form of uploaded electronic files (e.g., PDF, images, QR Codes) containing information about and copies of passports, government issued ids, travel documents, medical cards, health forms, and others. Participant personal data 307 may also comprise an exit method 669 and/or an exit reason 710. To maintain security and data privacy, when electronic files with personal information are uploaded, preferably they are stored in heavily restricted logical storage locations (e.g., AWS S3 buckets, Azure Blob Storage) encrypted using encryption keys, and identifiable only via one or more pseudonyms, which may include a participant id, a unique identifier 12, and similar data security constructs. Preferably, all data 10, including files (e.g., consent forms, passports, expense receipts) are stored into database 2 and associated with an individual unique identifier 12, for example as illustrated in FIG. 23. In addition, each record of data 10 may also be associated with a unique identifier 12 of a participant 26, facilitating scrubbing of personal information. Participant personal data 307 may further comprise traveler contact information 308, such as an address, phone, email, primary contact (e.g., the participant, caretaker), and contact instructions (contact by method, e.g., phone, email, SMS, and the appropriate contact number or address).

Traveler profile 309 may comprise data about traveler origin location 314, traveler identification 311, traveler health restrictions 312, and traveler preferences 313. Traveler origin location 314 may identify a street address for a participant (e.g., a home address, family home, hospital, facility, government building) as the preferred origin location for a participant's travel for a clinical study 25. Traveler origin location 314 also may comprise different traveler origin locations 314 depending on the mode of transportation, for example, a street address for car service pick up or drop off, or the traveler origin location 314 may be a preferred departure airport for flights according to a traveler flight preferences 341, a preferred bus terminal, train station, or port according to bus preferences 342, rail preferences 343, or ferry preferences 344, respectively. Traveler personal identification 311 may comprise a name, contact information 308, participant documents 310, known traveler number, and other information needed to book a flight, a hotel, reserve a car, or travel internationally.

Traveler health restrictions 312 comprises information about needed travel accommodations based on the traveler's health, medical conditions, and personal characteristics. For example, traveler health restrictions 312 may comprise information about a traveler's ability & assistance requirements, if a traveler has a vision or hearing impairment, does not speak the local language, requires a wheelchair or another assistive device, has cognitive or developmental disabilities, requires animal assistance, and others. Traveler health restrictions 312 may also comprise notes to assist a concierge user while booking travel.

Traveler preferences 313 may comprise information about travel and lodging preferences or requirements, including flight preferences 341, hotel preferences, car service preferences 345, car rental preferences 346, bus preferences 342, rail preferences 343, ferry preferences 344, hotel preferences 347, long-term housing preferences 348, and other travel preferences 313. Traveler preferences 313 may also comprise information about ambulance use and other conditions, such as supplemental oxygen, feeding tube, a colostomy bag, and others.

Flight preferences 341 may comprise information about preferred departure airports and airlines, seats, governmental security programs (e.g., TSA, known traveler numbers), and frequent flyer programs. Flight preferences 341 may also comprise information about allergies, need for an additional seat or for wheelchair assistance on and off the plane, travel with an oxygen tank, and other ADA requirements or restrictions, and other information about travel circumstances.

For example, bus preferences 342, rail preferences 343, and ferry preferences 344, may comprise information about a preferred departure port or station, transportation provider, and type/class of seating; boarding assistance needs; rewards programs, and other personal and health information. Car service preferences 345 and car rental preferences 346 may comprise information about a preferred service or rental company, vehicle class, pick up or drop off locations; wheelchair or other assistive needs, child safety seat requirement; and, other information about traveler's abilities and preferences. Lodging preferences, such as hotel preferences 347 or long-term housing preferences 348 may comprise information such as a preferred hotel, room floor, type and size, wheelchair accessibility, allergen free, rewards programs, parking, need for service animals, and any other ADA requirements or restrictions.

Companion dataset 325 may comprise information about travel companions (e.g., caregivers, guardians) of a participant. A travel companion may be a participant associated with a participant dataset 300, or a non-participant associated with participant companion dataset 300, comprising information necessary for travel.

Trip dataset 400 about a traveler's 26 trip may comprise a trip origin 401, trip destination 402, trip schedule 403, trip segment data 501, itinerary 475, and booked travel data 450. Booked travel data 450 may comprise segment booking information 508 for booked, reserved, or confirmed trip segments, and also may comprise traveler identification 311. Booked travel data 450 may also comprise price quote information for trip segments 501. Booked travel data 450 may vary based on trip segments. For example, for a flight segment booked travel data 450 may comprise a ticket and flight number, record locator, cost, airports, airlines, seating, schedule, customs and immigration information, luggage limitations, security warnings and requirements, and other air travel data. Hotel segment booked travel data 450 may comprise a hotel name and address, reservation number, cost, room type and amenities, hotel rules, policies, and other hotel information. An appointment segment booked travel data 450 may comprise visit (or appointment) confirmation, appointment schedule 136, appointment procedure 135, instructions such as direction to appointment location, access codes, preparation, and other information.

FIGS. 27, 32-35 illustrate an exemplary itinerary 475 comprising one or more of an itinerary message 478, booked travel data 450, trip segment data 501 (including segment booking information 508), traveler identification 311, traveler contact information 308, and other data from trip dataset 400 or participant dataset 300.

Trip origin 401 may comprise information about a trip's initial departure location, and may be based on (or determined according to) a traveler origin location 314, study travel policies 150, and a beginning trip segment data 501 (e.g., a trip's first trip segment). For example, if the beginning trip segment is a car service segment 514, trip origin 401 may be a street address provided in traveler origin location 31. In another example, if a trip requires air travel and study travel policy 150 does not permit a car service, the beginning trip segment may be a flight segment 512 and trip origin 401 may be a preferred departure airport in traveler origin location 314. Trip destination 402 may comprise information preferably based on a site appointment location 201 including address, directions, and other requirements. When a trip dataset 400 comprises one trip segment, such as an appointment segment 511, travel origin 401 and travel destination 402 may be the same (e.g., correspond to a site appointment location 201).

Trip schedule 403 may comprise data about one or more of a departure time, a duration, and a return time (e.g., time of departure for the return trip). Trip schedule 403 preferably is based on one or more of appointment schedule 136 and trip segments 501. For example, trip schedule 403 departure time may be determined based on appointment schedule 136 and outbound trip segment schedules 505 so that a participant arrives at trip destination 402 before or at the appointment schedule 136 appointment start date and time, or in some embodiments, before an arrive-by-date-and-time 137. In another example, trip schedule 403 return time may be the departure time for a return trip and may be determined based on appointment schedule 136 so that a participant departs appointment location 201 after the appointment end date and time, or after the depart-after-date-and-time 138.

Trip segment data 501 may be data about an appointment segment 511, a flight segment 512, a bus segment 513, a car service segment 514, a car rental segment 515, a rail segment 516, a ferry segment 517, an ambulance segment 518, and a lodging segment 519. Embodiments may comprise a trip dataset 400 having a first trip segment data 501, for example, an appointment segment 511, and a second trip segment data 501, for example a flight segment 512.

Trip segment data 501 (or trip segment 501) may comprise information about a segment start location 503, segment end location 504, segment schedule 505, segment status 506 (e.g., booked, booking requested, pending, confirmed, reserved, etc.), segment instructions 507, segment booking information 508, and information. Segment instructions 507 may comprise messages (e.g., allergies, health and medical needs, preferences) to the trip segment provider (e.g., airline, car company, hotel) based on the traveler profile 309 (e.g., traveler health restrictions 312, traveler preferences 313), as well as information/directions for a traveler (from, e.g., clinical site dataset 200).

Trip segment data 501 may be determined (by system 1 or a user) based on data from traveler profile 309, study travel policies 150, visit schedule 130, clinical site dataset 200, and a previous or subsequent trip segment 501. For example, a beginning trip segment start location 503 may be the trip origin 401, or the segment start location 503 for the first trip segment 501 of a return leg of a trip may be the trip destination 402 (e.g., appointment site location 201). In another example, a first segment end location 504 may be based upon (e.g., same as, nearby) a second segment start location 503 of a second (e.g., subsequent to the first) trip segment 501. Similarly, a second trip segment start location 503 may be based on, or be the same as, the first segment end location 504. Trip segment data 501 may comprise maps or directions (e.g., from google maps, street view, hotels or clinical site photos) related to trip segment start location 503, trip segment end location 503, and data from previous or subsequent trip segment data 501.

Trip segment schedule 505 (or just segment schedule 505) may comprise a segment start time (e.g., departure time, check in time), segment duration, and/or a segment end time (e.g., arrival time, check out time). For example, a first segment schedule 505 (e.g., start time, end time) may be based upon a second (e.g., preceding or following the first) segment schedule 505 (e.g., end time, start time). Beginning trip segment schedule 505 and the initial trip segment 501 of a return leg of a trip may be based on the trip schedule 403 departure time and trip schedule 403 return time, respectively.

Segment booking information 508 comprises information necessary to book or reserve a trip segment determined based on traveler profile 309 (e.g., traveler health restrictions 312, traveler preferences 313), and applicable study travel policy 150 (e.g., maximum cost, permitted trip segment or services classes, etc.). Segment booking information 508 may also comprise information for a traveler based upon booked travel data 450 (e.g., flight, bus, or train numbers; reservation number; rental car model, color or license plate; ambulance provider; seat numbers; hotel name), clinical site dataset 200 (e.g., site contact, access instructions), and other helpful information.

Appointment segment 502, comprises data about a patient visit to a clinical site for clinical study assessment, and may be determined based on study visit schedule 130 and/or clinical site dataset 200, including, for example, appointment schedule 136, appointment procedure 135, and appointment location 201.

Flight segment data 512 may be a one way flight segment 512 having an outbound flight 512a, or a round trip flight segment 512 having an outbound flight 512a and a return flight 512b. Flight segment booking information 508 may comprise information necessary to book all flights in a flight segment 512 determined based on data from one or more of traveler profile 309, including for example, flight preferences 341, study travel policy 150, and traveler identification 311. Segment booking information 508 for a flight segment 512 may also comprise data from booked travel data 450, including airports, terminals, flight numbers/carriers, confirmation/ticket numbers, baggage information, and other flight information.

Flight segment start location 503 may be determined based upon traveler profile 309 (e.g., preferred airport, distance from traveler home address), or segment end location 504 of a preceding trip segment 501 (e.g., a rail segment 516, another flight segment 512, appointment segment 511). Flight segment 512 segment end location 504 may be based upon traveler profile 309 and/or a subsequent travel segment (e.g., appointment location 201, lodging segment 513 location, bus segment start location). A round trip flight segment start location 503 may be the same as the round trip flight segment end location 504 (e.g., same arrival and departure airport). In that example, outbound flight 512a end location 504a may be the same as return flight 512b start location 503b. Flight segment schedule 505 may comprise flight departure and arrival times, as well as flight durations, for all connecting outbound and/or return flights 512a, 512b, of a flight segment 512.

Lodging segment 513 may be a hotel segment 513 or long-term lodging segment 513. Lodging segment booking information 508 may comprise room type, floor, size, accessibility, preferred or booked hotel or lodging company, lodging confirmation and/or reservation numbers, and other information based on traveler profile 309 (e.g., hotel preferences 347, long-term housing preferences 348), study travel policy 150, and booked travel data 450. Lodging segment start location 503 (preferably the same as end location 504) comprising a lodging address, may be based upon traveler profile 309 (e.g., preferred hotel), distance from a preceding or subsequent trip segment (e.g., appointment location 201, arrival or departure airport, etc.). Lodging segment schedule 505 (date/time to, e.g., check in and check out times) may be determined based on appointment schedule 136, traveler profile 309, travel policy 150, and preceding or subsequent trip segments (e.g., arrival and departure dates/times).

Surface transportation trip segments 501 such as rail segment 343, bus segment 342, and ferry segment 344, may comprise data about a one way or round trip surface transportation, including, surface transportation segment booking information 508 (e.g., train, bus, ferry, or route numbers, transportation company, confirmation/ticket numbers, baggage information) and other information based on one or more of traveler profile 309 (e.g., rail preferences 343, bus preferences 342, ferry preferences 344), study travel policy 150, and booked travel data 450. Surface transportation segment start location 503 may be determined based on traveler profile 309 (e.g., preferred station or port, distance from traveler home address), previous segment end location 504 and other information. Surface transportation segment end location 504 may be based on traveler profile 309 and clinical site dataset 200 (e.g., appointment location 201, arrival instructions). Surface transportation segment end location 504 may be based on a subsequent trip segment start location 503. Segment schedule 505 of a surface transportation trip segment 501 may comprise departure and arrival times and segment durations, for all connecting outbound and/or return train, bus, or ferry routes within a surface transportation trip segment 501.

Car service segment 514 and car rental segment 515 (also referred together as “car segment 514, 515”) booking information 508 (e.g., vehicle number/license plate, car company, confirmation numbers, luggage information) may be based on one or more of traveler profile 309 (e.g., car service preferences 345, car rental preferences 346), study travel policy 150, and booked travel data 450. Car segment start location 503 (e.g., pick up location) may be determined based upon traveler profile 309 (e.g., traveler origin location 314), or a previous trip segment end location 504 (e.g., airport, appointment location). Car segment end location 504 (e.g., a drop off location) may be determined based on a traveler's home address, or on a subsequent trip segment start location 504 (e.g., airport, appointment location). Segment schedule 505 of a car service segment 514 may comprise pick up and drop off times, as well as trip duration.

Travel planning interface 500 of GUI 43 provides an easy and efficient process for a travel coordinator (e.g., user, concierge user) to plan and book a trip for a traveler. GUI 43 may be configured to switch to the travel planning interface 500 by utilizing controls as described above.

Travel booking module 575, illustrated in FIG. 1, may be any type of system, software, device, entity, or combinations thereof, configured to i) receive booking request data 451 about a trip or trip segments from travel planning interface 500, ii) book, or obtain a quote for, the trip or trip segment based on the booking request data 451; and iii) transmit or provide to travel planning interface 500 booked travel data 450 about the trip or trip segment. Booking request data 451 comprises one or more of trip segment data 501, including segment booking information 508 (for each trip segment 501, e.g., desired start and end locations 503, 504, schedules 505, traveler restrictions and preferences) determined during travel booking using travel booking interface 500, and traveler identification 311 needed to obtain a booking (e.g., for flights, international travel, hotels, etc.).

Travel booking module 575 may comprise a browser interface that may be separate or distinct from GUI 43 and/or travel planning interface 500, may be networked with GUI 43, or may be incorporated (e.g., as a separate panel, interface, frame) within GUI 43 or within travel planning interface 500. Such travel booking module 575 may be configured to enable a user to access travel websites, and search for, select, and/or book, appropriate trip segments (e.g., flight, train, bus, hotel, car rental, car service) based on booking request data 451. Travel websites may include website of travel providers, such as airlines, railroads, bus companies, hotels, car rentals or service companies, ambulance operators, and others, or may include travel booking websites (e.g., booking.com, orbitz.com, travelocity.com, expedia.com, hotels.com, carrentals.com). Travel booking module 575 may also comprise, or include the services of, virtual or conventional travel booking agents (e.g., independent or affiliated with a travel company), including general travel agencies, agencies specializing in a particular type of transport (e.g., car service, long-term housing, ambulance, medical transportation, etc.), and other types of booking agents. Travel booking module 575 may also comprise an interface to a clinical site (e.g., a clinical site scheduling personnel or system) to book (e.g., confirm) an appointment segment 511.

Travel booking module 575 preferably will book, or obtain quotes (automatically and/or with human involvement), for a trip or trip segment based on received booking request data 451 (e.g., a flight between requested airports on a requested date, requested hotel reservation for a duration appropriate for traveler restrictions), and transmit to travel planning interface 500 booked travel data 450 including booking or quote information about the trip or trip segment. Segment booking information 508 may be updated with booked travel data 450 received from travel booking module 575.

Booking request data 451 may be transmitted by travel planning interface 500 electronically over a network (using, e.g., API, URL incorporating trip segment information, data files with trip information, email), by telephone, or from input device 41 (e.g., when travel module 575 is a travel booking website). Embodiments of travel planning interface 500 may also transmit booking request data 451 by storing it in database 2, and causing system 1 (e.g., servers 3) to access booking request data 451 in database 2 and send it to travel booking module 575, for example, using the above described methods. Travel planning interface 500 may receive booked travel data 450 similarly over a network, by email, in the form of printouts, from database 2 (e.g., if servers 3 received and stored booked travel data 450).

GUI 43 provides efficient access to travel planning interface 500 from various views and panels by utilizing, for example, controls 483, 485 associated with a participant 26 (from, e.g., individual trip module 59, participant interface 82, etc.). Travel planning interface 500 comprises trip segment controls and trip segment panels 520 that can be utilized to correct, enter, customize, and confirm information in trip segment data 501 for each trip segment and for each participant, for example by personalizing trip segment data 501 for each participant, adding notes, detailed directions and instructions, and personalized requests.

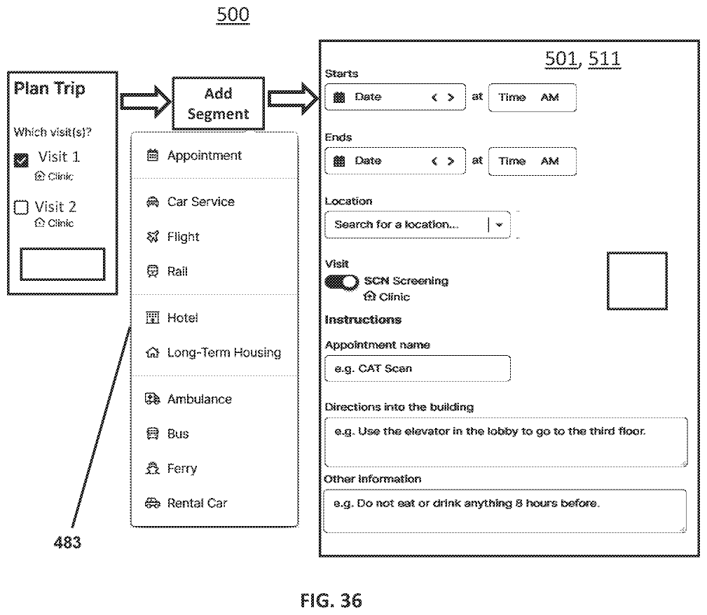

An embodiment of the invention may be utilized and function as illustrated in the flowchart in FIG. 4. In step 1000 a user selects a participant 26 utilizing control 485 (or 482), as indicated in step 1001, to initiate trip planning interface 500. Trip planning interface receives from database 2 a trip dataset 400 associated with patient 26 and participant visit data 304. In step 1002, current visit 306 from patient visit data 304 may be displayed for confirmation (also allowing a selection of a different current visit 306 prior to confirmation). Upon confirming a current visit 306 the travel interface 500 advances to step 1003 and the GUI switches to a sequence of segments panels 520 providing controls 483 allowing the addition of a trip segment 501. In step 1004 another trip segment 501 may be selected using control 483 (e.g., selecting a flight, car service, hotel, or other segments from a menu) as illustrated in FIG. 36, or control 483 may be used to indicate that no more segments will be added (e.g., clicking “finish”). Depending on study travel policy 150 applicable to the trip or patient 26, some types of trip segments may or may not be available. For example, if the main travel policy 151 does not allow bookable car service, car service segments may not be able to be booked (or may require approval), unless a specialized travel policy 152 applies (e.g., country policy based on origin or destination, visit policy, etc.) and allows car service. In such an example, if patient 26 or current visit 306 are subject to a specialized travel policy 152 (based on, e.g., patient country, visit 306 type, etc.) car service segment may be available for selection in step 1004.

If a new segment is selected, indicated with “yes” in step 1005, travel planning interface 500 advances to step 1006 displaying the appropriate trip segment panel 520 (e.g., flight segment panel, hotel segment panel, appointment segment panel, etc.) with appropriate trip segment data 501. Trip segment data 501, and preferably segment booking information 508, may be determined as described above based on traveler profile 309, study travel policies 150, current visit 306 (e.g., appointment procedure, schedule), clinical site dataset 200 (e.g., appointment location associated with the appointment procedure), and trip segments data 501 for previous or subsequent trip segments that have already been generated for the trip using travel planning interface. Trip segment data 501 may also be determined based on study travel policies 150 applicable to patient 26 and current visit 306. For example, if patient 26 is associated with a traveler label 321 (e.g., first class allowed) a flight trip segment data may include information about first class tickets; or if current visit 306 is associated with a visit travel policy (based on, e.g., a visit type or id, or visit label 131) that allows long-term lodging, lodging segment data may include information about long-term lodging.

Trip segment panel provides controls that may be utilized to modify, select, input, and confirm trip segment data 501. In step 1007, the travel planning interface 500 enables a user to “request booking” by transmitting to travel booking module 575 booking request data 451, comprising trip segment data 501, preferably including segment booking data 508, determined in step 1006 for a trip segment selected in step 1004.

After transmitting booking request data 451 to travel booking system 575 the travel planning interface 500 returns to step 1004, as indicated by arrow 1008, allowing the selection of another trip segment 501 or to finish planning the trip. If another trip segment is selected, travel booking interface 500 repeats steps 1005 1007. If no more trip segments are needed, indicated by a “no” in step 1005, the travel interface 501 advances to step 1009 to receive the booked travel data 450 from travel booking module 575. Embodiments may be configured to transmit booking request data 451 for each trip segment 501 in step 1007. Other embodiments may be configured to include an aggregation step (not shown) where booking request data 451 are aggregated for all trip segments 501, and transmitted as booking request data 451 for an entire trip.

Embodiments may be configured with an additional check for travel policy compliance, indicated by steps 1020-1023, preferably when a system 1 is configured to permit non-compliant travel upon approval. After a confirmation of trip segment data 501 in step 1006, the in step 1020 a computing algorithm compares trip segment data 501, and preferably segment booking information 508, to the applicable study travel policy 150 (e.g., cost, whether certain services are bookable or not, and other) including main travel policy 151, and any specialized travel policy 152 applicable to the trip being booked (e.g., based on individual traveler policy labels 321, countries, current visit 306, visit policy label 321, and others). If trip segment data complies with all applicable study travel policies 150, indicated as “YES” in step 1021, the travel interface 500 continues to step 1007. If the algorithm results in non-compliance (e.g., “NO” in step 1021), travel planning interface 500 invokes an approval workflow 1022 (which may involve escalation to management, or other steps). If the approval workflow 1022 results in approval in step 1023 (“YES”), the travel interface 500 advances to step 1007. If workflow 1022 results in a rejection in step 1023 (“NO”), travel interface 500 returns to step 1006 allowing the user to modify or input new trip segment data.

In step 1009, trip planning interface 500 may generate an itinerary 475 as described above (and including received booked travel data 450), and may enable GUI 43 to switch to individual trip 59 module for further actions, such as sending notifications, entering additional trip information, or modifying travel segments.

An embodiment of the invention is a system for visualizing and managing a clinical study 25 schedule of assessments (also referred to as a visit schedule, a schedule of events, a schedule of visits, and others) for a participant 26, or for all participants 26. A visualization aspect of various embodiments may also facilitate creation of detailed schematics of a study 25 design describing all visits and assessments. Embodiments may represent a schedule of assessments for a clinical study 25 as a study visit schedule 130 in data 10 wherein study visit schedule 130 is associated with participants 26 in a study 25 and comprises data about participants 26 visits 610 (or appointments 610). Each appointment or visit, as well as other events and steps according to a clinical protocol schedule of events may be represented as a data node 600, or simply a node 600, in visit schedule 130. For example, a visit node 600 may represent an appointment 610 or visit 610, a cycle node 600 may represent a visit cycle 650, a jump node 600 may represent a visit jump 636, a track selector node 600 may represent a track selection decision point 627 at which patients may be assigned to study arms or clinical groups (here, referred to as “tracks,” or “visit tracks”), an exit node 600 may represent a track exit 665 at the end of a track 700, at which point a decision must be made how a patient will exit the study according to exit reasons 710. Information node 607 and verification nodes 606 may provide information relevant to the study or subsequent visits, or instruct that a patient's identity be verified before proceeding with subsequent visits. Additional nodes 600 serving different functions or purposes may also be provided to facilitate the management of patients in the study.

The nodes may be grouped into one or more tracks 700, 701 (e.g., a group, a series, or a sequence of events or steps represented by nodes) with each track 700, 701 comprising one or more nodes 600. Track 700 may be a study track 700 (representative of sequence of events applicable to, e.g., a study arm or group, a patient subgroup, and others) or an exit track 700 (e.g., representative of sequences of pre-exit/pre-termination events). The nodes 600 in each track 700 may be ordered in a sequence of steps (a “step sequence”) 702. Track 701 may be an unscheduled visits track 701 comprising one or more nodes 600 for unscheduled visits 611.

Each node 600 may comprise a node position 601 indicating the location of the node within the visit schedule. The node position 601 may comprise a node track 603 and a node step 602. The node track 603 comprises information to identify one or more tracks 700, 701 with which node 600 is associated. Node step 602 contains information (e.g., a number, character, link, memory address, alphanumeric value, etc.) corresponding to a location in track 700, 701 identified by node track 603, and a step in step sequence 702 of track 700, 701. Preferably, nodes 600 are sequentially ordered in a step sequence 702 according to node step 602 of each node 600. Node track 603 may comprise a home track 604 identifying a parent (or source) track 700, 701 of a visit node 610, a cycle node 650, a track selector node 627, an information node 600, and a verification node 600. Node track 603 may also comprise a jump start track 639 and a jump destination track 642 identifying a track 700 where a jump starts, and track 700 where the jump ends according to jump destination 641, respectively.

A node step 602 may represent a visit step 602 in a visit node 610, a track selector step 602 in a track selector node 627, and an exit step 602 in an exit node 665. Preferably, track selector step 602 and exit step 602 are a last step in a step sequence 702 of a track 700. The node step 602 of a jump node 636 may comprise a jump start step 640 and a jump destination step 643; and a node step 602 of a cycle node 650 may comprise a cycle start step 653 and a cycle end step 652.

The node position 601 may be a visit position 601 comprising a visit step 602 and a home track 604; a cycle position 651 comprising a cycle start step 653, cycle end step 652, and a home track 604; a track selector position 628 comprising a track selector step 602 and a home track 604; an exit position 601 comprising an exit step 602 and the home track 604; and a jump node position 637 which may contain a jump start position 638 comprising a jump start track 639 and a jump start step 640 (e.g., in the jump start track), and a jump destination 641 comprising a comprising a jump destination track 642 and the jump destination step 643 in the jump destination track 642.

Each node 600 may also have node attributes 605 with information about various aspects and characteristics of the node. For example, node attributes 605 of each node 600 may comprise a node data id 12 and a node type (e.g., visit, cycle, jump, track selector, track exit, information, verification). Node data id 12 may be a data id 12 that may be used to enable GUI 43 or visit schedule interface 80 to securely access node 600 information from database 2, and may also be used to associate node 600 with data records in data 10, for example visit schedule 130, participant dataset 200, Clinical site dataset 300, trip dataset 400, and others.

Node attributes 605 may be visit node attributes 613, cycle node attributes 654, jump node attributes 644, track selector node attributes 630, exit node attributes 668, information node attributes 605, and verification node attributes 605.

Visit node attributes 613, may comprise a visit id 614, visit name 615, visit description (or purpose) 622, study period 103, Appointment Schedule 136 (also referred to as timing in the figures), visit duration 621, visit form 620, visit policy label 131, and other visit data node attributes.

Visit id 614 (e.g., Visit_A2, Visit 0) may comprise a visit identifier 616 (e.g., V or Visit, PL or preliminary, A or Assessment, F or Follow-up, BL or Baseline, SCR or Screening, SRG or Surgery, etc.) and a visit number 617 to indicate the visit's sequential order among other visits with the same visit identifier 616 (e.g., Visit0, Visit1, BL1, BL2). Visit name 615 may describe a visit, a visit purpose, a study period, an appointment procedure 135, or others (e.g., preliminary, screening, surgery, radiology assessment). Visit description 622 preferably identifies the purpose of a visit, such as an appointment procedure 135, and may also include additional details, including for example, a description reflecting specimens to be collected, specific imaging to be performed, specific laboratory test or assays needed, patient questionnaires, and other details. Allowed visit form 620 identifies whether the appointment procedure may be performed in a clinic, at home, or virtually.

Study period 103 is representative of periods during the study visit schedule (e.g., screening, enrollment/baseline, follow-up, early termination, etc.). Appointment Schedule 136 may comprise a visit time window 619 previously described. Visit duration 621, indicating how long a visit is expected to take (generally for scheduling and travel purposes), may be provided, or may be generated based on visit name 615, study period 103, appointment procedure 135 and/or other factors.

Cycle node attributes 654 comprise a cycle end condition 655 and cycle timing 658. Cycle end condition 655 may be a manual end condition 655 (represented as, e.g., unlimited repetitions, until manually interrupted) or end after a number of repetitions condition 655 (e.g., a number, count, quantity of repetitions or a range thereof; or end after “at least but no more than,” or “at most,” repetitions providing for manual termination after a minimum number of repetitions). Cycle timing 658 indicates when the next (or new) repetition of cycle 650 will start and may indicate a time window 658a (e.g., “1 hour,” “3 days”) as well as whether the time window will be counted from the end or from the start of the previous repetition. In other embodiments the next repetition of cycle 650 may be configured to start a number of days (or other periods) from the end or the start of the previous repetition, plus/minus time window 658a. Jump node attributes 644 comprise a jump optional indicator 645 if a jump is not required.

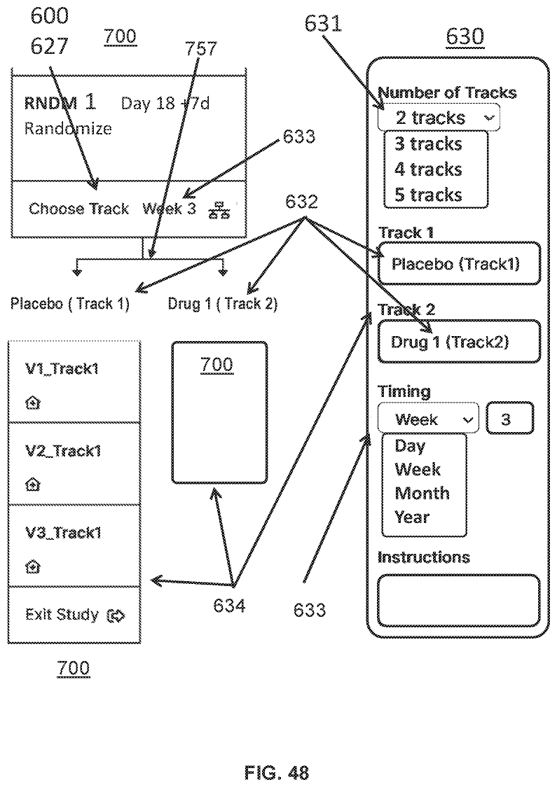

Track selector node attributes 630, exemplified in FIG. 48, may comprise a number (or quantity) of destination tracks 631 indicating how many new destination tracks 634 need to be generated in the data of the visit schedule 130. Track selector node attributes 630 also comprise a destination track name 632 for each new destination track, and preferably comprises as many destination track names 632 as the number of destination tracks 631. Track selector node attributes 630 also may comprise track selection timing 633 indicating when the track selection should occur (e.g., the assignment of patients to different tracks). Track selection timing 633 may be relative or absolute. Track selector node 627 allows additional tracks to be created in the database 2 and visualized by the visit schedule interface 80, where each track may correspond to a study arm or clinical subgroup described in the clinical protocol.

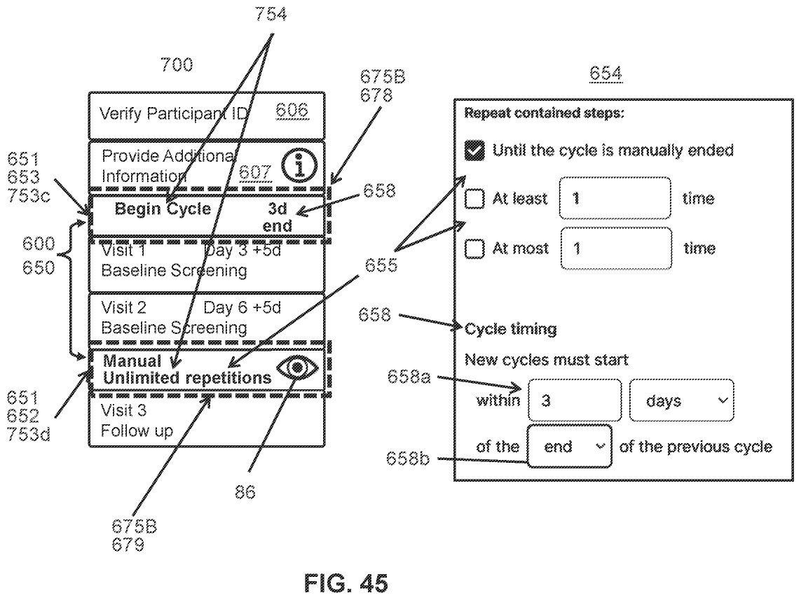

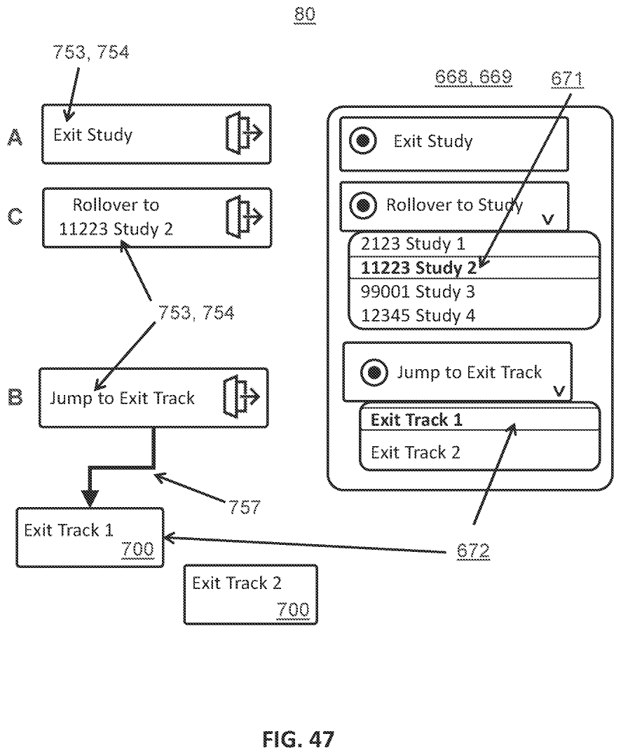

Exit node attributes 668 may comprise an exit method 669 indicative of how a patient who has reached the exit node 665 should proceed as illustrated in FIG. 47. Exit method 669 may indicate for patient 26 one of “Exit Study,” “Jump to Exit Track,” and “Rollover to Study.” Exit method 669 may further comprise a destination exit track 672 identifying an exit track 700 (e.g., “Exit Track 1”) to which a patient should be assigned and a rollover study id 671 (a clinical study identifier) in which to enroll patient 26. Information node attributes (not shown) may comprise information about the study, track, and particular visit. Node attributes 600 for a Patient Verification 606 may comprise instructions on verifying a patient's 26 identity as illustrated in FIG. 45.

Each track 700, 701 may further comprise a track id which may include a track data id 12 used to uniquely and securely identify and access track 700, 701, for example by the visit schedule interface 80, the database 2, and other internal or external components or systems. The track id (not shown) may also comprise a track name 632.

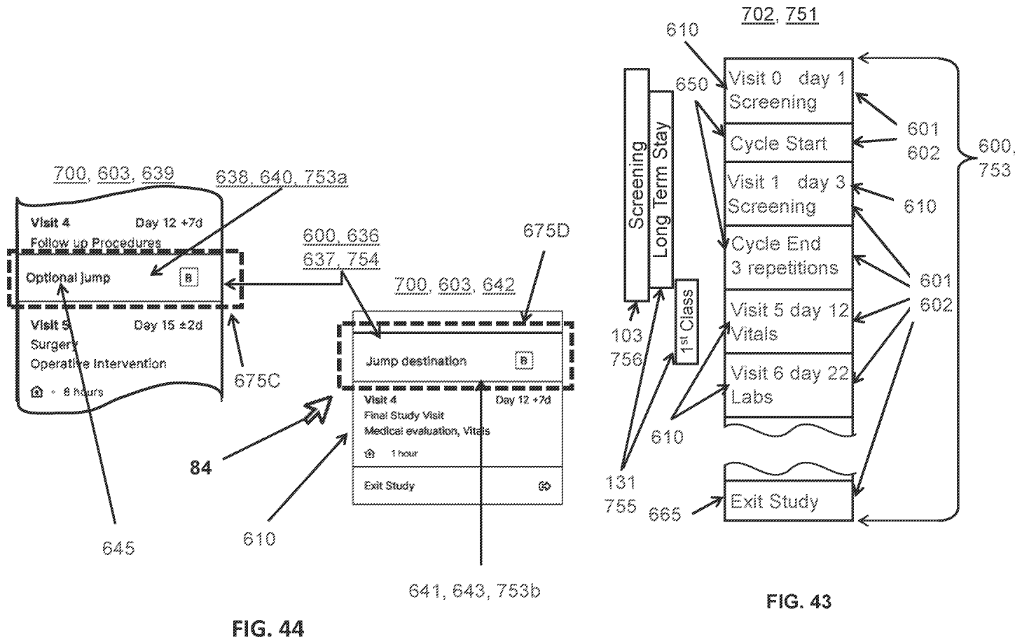

As exemplified in FIG. 43, track 700 may comprise a step sequence 702 of nodes 600 sequentially ordered according to the node step 602 of each node 600. It should be understood that track 700 and step sequence 702 may be represented in the data in various different ways. For example, a dataset may represent each track and store node data ids and/or node steps 602 of nodes associated with the track and the data of each node may contain information about the associated track and/or the node step 602. Various other methods, as well as combinations of the preceding and other methods are well known in the art and are not described herein.

An exit track 700 step sequence 702 comprises nodes representing final procedures/evaluations that should be performed before a patient exits the study and may depend on the reason for a patient exit, and whether the exit is an early termination or not. As illustrated in FIG. 50, exit track 700 may further comprise an exit track name 632 and information indicating exit reasons 710 associated with exit track 700, for example, death, failing screening, medical disruption, side effects, pregnancy, unresponsive to interventions, rolling over to another study, patient withdrawal, and others. Exit reason 710 may also be indicated as unknown. Embodiments may be configured so that a patient who is exiting the study will be assigned to an exit track based on a patient's 26 reasons for exiting 710.

The visit schedule interface 80 displays a track graphic 750 visualizing a track 700, 701, for example a step sequence 702 of track 700, or the grouping of nodes in an unscheduled visits track 701. Track graphic 750 may comprise a step sequence graphic 751, a track header graphic 752, and a track connector graphic 757. When visit schedule 130 comprises two or more tracks 700, 701, visit schedule interface 80 may display two or more track graphics 750 to visualize each of tracks 700, 701. A step sequence graphic 751, visualizing a step sequence 702 of a track 700, may comprise one or more selectable node graphics 753, wherein each selectable node graphic is associated with, and represents, one node 600 associated with track 700. Track header graphic 752 may be representative of the track name 632 and other track attributes. A track graphic for an exit track may comprise a selectable exit track header 752 indicating the exit reasons 710 applicable to the exit track 700. Selectable exit track graphic 752 may be associated with control 85, which may be activated upon selecting exit track graphic 752.

Visit schedule interface 80 may also display a travel policy graphic 755, and a study period graphic 756, each of which may be configured to be selectable. Travel policy graphic 755 indicates that a node (e.g., a visit) has a visit policy label 131 attribute associating it with a visit travel policy 153 of Study Travel Policy 150. Study period graphic 756 indicates the study period 103 during which a node (e.g., a visit) occurs.

Preferably, in each track graphic 750, the selectable node graphics 753 are displayed sequentially in the same order as their associated nodes 600. FIGS. 45, 46, 47 illustrate embodiments in which each selectable node graphic 753 comprises visual attributes 754 (e.g., text, images, icons, graphics, etc.) representative of node attributes 613 of node 600 associated with the selectable node graphic 753.

Visual attributes 754 may be selectable when the selectable node graphic is selected, or a visual attribute 754 may be independently or individually selectable. For example, selectable node graphic 753 may comprise an individually selectable visual attribute icon 754 associated with a user control, preferably one or more of an information control 86, a visit schedule control 83, and a selection control 84.

For example, a selectable node graphic 753 associated with a visit node 600 may display one or more visual attributes (in the form of, e.g., color, shading, fonts, text, lines, geometric shapes, icons, images, etc.) identifying node 600 as a visit 610, and representative of visit node attributes 613 such as visit id 614 (e.g., V0, ASMNT1), visit name 615 (e.g. Visit 0, Assessment 1, Follow-up Assessment), duration 621 (e.g., “1 hour”), Appointment Schedule 136 and/or visit time window 619 (e.g., “floating,” “day 3+7d”), Visit Description 622, allowed visit form 620 (illustrated in the figures as a clinic icon for a clinic visit, a telephone for a virtual visit, and of a house for home visits.) Visual attributes 754 for a verification node 600 may comprise instructions to verify a participant's identification before proceeding to the next visit 610. Visual attributes for an information node 600 may comprise additional relevant information.

As illustrated in FIG. 47, a selectable node graphic 753 for a track exit 665, may comprise visual attributes of text or graphics indicating the exit method 669, for example “Exit Study” indicated with the letter “A,” Rollover to 11223 study” indicated with the letter “B,” and “Jump to Exit Track 1”) indicated with the letter “C” and a track connector graphic 757.

FIG. 44 illustrates an embodiment of a selectable node graphic 753 representation of a jump node 636 comprising jump visual attributes 754, a selectable jump start graphic 753a associated with the jump start position 638, and a selectable jump destination graphic 753b associated with the jump destination 641. The jump visual attributes may comprise an “optional jump” indicator, and a jump identifier (e.g., jump “A”, jump “B”), to visually associate a jump start and a jump destination as part of the same jump, illustrated in FIG. 44 as squares with an inscribed letter “B”). FIG. 45 illustrates a selectable node graphic 753 associated with a cycle graphic, and comprising cycle visual attributes 754, a cycle start graphic 753c associated with the selectable cycle start step 653, and a selectable cycle end graphic 753d associated with the cycle end step 652. The cycle visual attributes may indicate the cycle end condition 655 (repeat, e.g., “unlimited,” “4-5” times), and cycle timing 658 (e.g., “5d/end” indicating 5 days from the end of the previous repetition).

A selectable node graphic 753 of a track selector 627 may comprise visual attributes indicative of track selection timing 633 (e.g., “week 3”) and a track connector graphic 757 connecting to destination tracks 634 to which patient 36 may be assigned.

Visit schedule interface 80 may comprise a visit schedule control 83, a selection control 84, exit track control 85, information control, and a book travel control. The book travel control, preferably available from the patient visit schedule interface 82, is configured to launch the travel interface 500 to book travel.

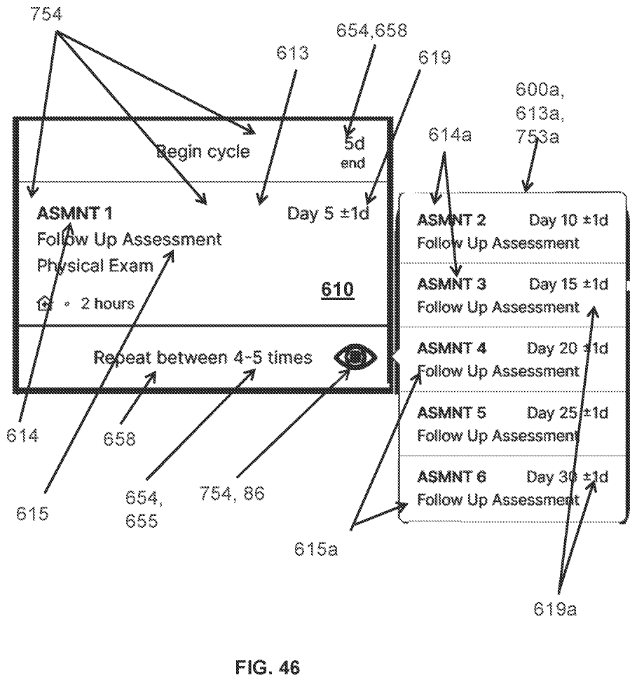

FIG. 46 illustrates information control 86, which may be associated with a selectable icon (shown as an Eye in the figure). Visit schedule interface 80 may be configured to enable using the information control 86 to display generated node information when a user selects the selectable icon. For example, as shown in FIG. 46, when the selectable icon (e.g., an eye) in node 600 of a cycle 650 is selected, visit schedule interface 80 may be configured to calculate (or in general cause another computing resource of system 1 to calculate, generated node attributes 613a and generated node graphics 753a based on the visit node attributes 613 and cycle node attributes 654 and display the generated node information. In this example generated nodes 600a with generated visit IDs 614a (e.g., “ASMNT 2,” “ASMNT 3,” “ASMNT 4,” “ASMNT 5,” “ASMNT 6”), generated visit names 615a (e.g., “Follow-up Assessment”) and generated visit time windows 619a (e.g., Day 10+/−1d, Day 15+/−1d, etc.) may automatically be generated (or calculated) based on a visit 610 in cycle 650, visit ID 614, visit name 615, visit time window 619, and cycle repetitions 658.

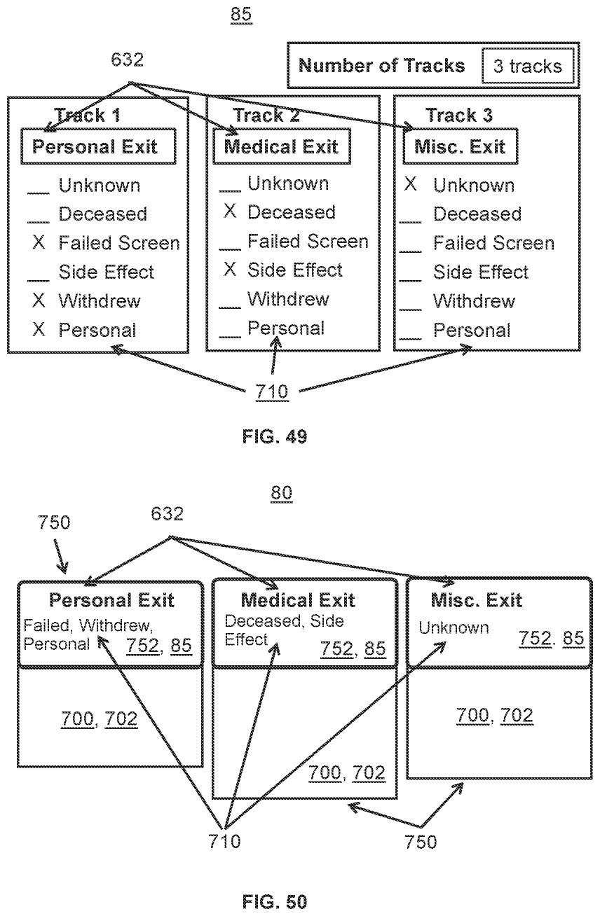

The Visit schedule interface 80 may be configured to enable using exit track control 85 to add, remove, and/or modify exit tracks 700 (e.g., change exit track names 632). Exit reasons 710 associated with exit track 700 may also be added and/or modified using control 85 as illustrated in FIG. 49. Exit track control 85 may be used to add or delete exit tracks 700, for example, by providing to visit schedule interface 80 (e.g., through input device) an indication to add one or more new exit tracks or an indication to delete one or more of the exit tracks. Exit track control 85 may provide an indication to add or to delete tracks by providing, and the visit schedule interface 80 receiving, a new quantity 709 of exit tracks to visit schedule interface 80. A new quantity 709 of exit tracks greater than the current quantity (e.g., number) of exit tracks 700 in visit schedule 130 may be an indication to add exit tracks and visit schedule interface 80 may be configured to add as many new exit tracks 700 as is the difference between the new quantity of exit tracks and the quantity of exit tracks in visit schedule 130. New quantity 709 of exit tracks lower than the current quantity (e.g., number) of exit tracks 700 in visit schedule 130 may be an indication to delete exit tracks and visit schedule interface 80 may be configured to remove as many exit tracks 700 as necessary so that the there remains a new quantity 709 of exit racks in visit schedule 130. In other embodiments exit track control 85 may be used to provide an indication to add or delete exit track by providing to visit schedule interface 80 a quantity of exit tracks 700 to add or delete. Exit track control 85 may further comprise inputs or controls that allow a user to specifically indicate to visit schedule interface 80 an exit track to be deleted. In response to using the exit track control 85, the visit schedule interface 80 may cause database 2 to generate and store data records for each new exit track reflecting the new exit track names 632 and exit reasons associated with each exit track. Further, the visit schedule interface 80 may generate and display a track graphic 700 associated with each exit track.

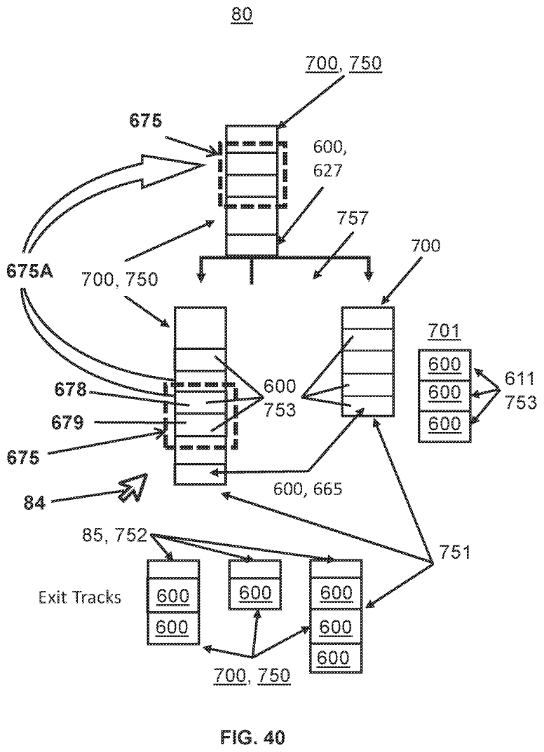

Visit schedule interface 80 is configured to enable using the selection control 84 (e.g., illustrated as a mouse cursor in the Figures) to select one or more nodes 600 as selected nodes 675 (e.g., indicated by a thick dashed line in FIG. 40) by selecting the selectable node graphics 753 associated with the selected nodes 675. The selected nodes 675, may be a single node, or may be a group of two or more nodes. The selected nodes 675 (or the data representative of the selected nodes 675) will comprise selection position 676, preferably containing a selection node track 603 (e.g., identifier of the home track 604 of the selected nodes 675), a selection start step 678 (e.g., the node step 602 of the first selected node), and a selection end step 679 (e.g., the node step 602 of the last selected node). Selection position 676 may also comprise information representative of the node positions 601 of all selected nodes 675. For example, if selected nodes 675 comprise a single node, e.g., representing a visit, track selector, information, verification, or exit, then the selection position 676 will be the same as the node position 601 of the node, and the selection start step 678, selection end step 679, and the node step 602 of the selected node will be the same. In another example, if the selected nodes 675 comprise a track selector node 627 the selection start step 678 and the node step 602 of the track selector node 627 will be equal.

In another example, if selection control 84 is used to select a cycle node graphic 753 the selected nodes 675 may comprise only the associated cycle node 650 (indicated on FIG. 45 as thick dashed boxes 675B), or may comprise all nodes between the cycle start step 653 and the cycle end step 652 (e.g., all nodes between dashed boxes 675B). In this example, the selection start step 678 and selection end step 679 may be equal to the cycle start step 653 and cycle end step 652, respectively.

In another example, the selection control 84 may be used to select a jump start graphic 753a or a jump destination graphic 753b and the selected nodes 675 may comprise one or both of the jump start position 638 and the jump destination 641, indicated as dashed boxes 675C, 675D in FIG. 44. Visit schedule interface 80 may be configured so that the selection position 676 comprises the jump node position 637, including the jump start position 638 and the jump destination 641. In another embodiment, visit schedule interface 80 may be configured so that the selection position 676 comprises one of the jump start position 638 and the jump destination 641, for example, based on whether the jump start graphic or jump destination graphic was selected first, based on whether the jump start position 638 or jump destination 641 occur earlier in time, and various other factors.

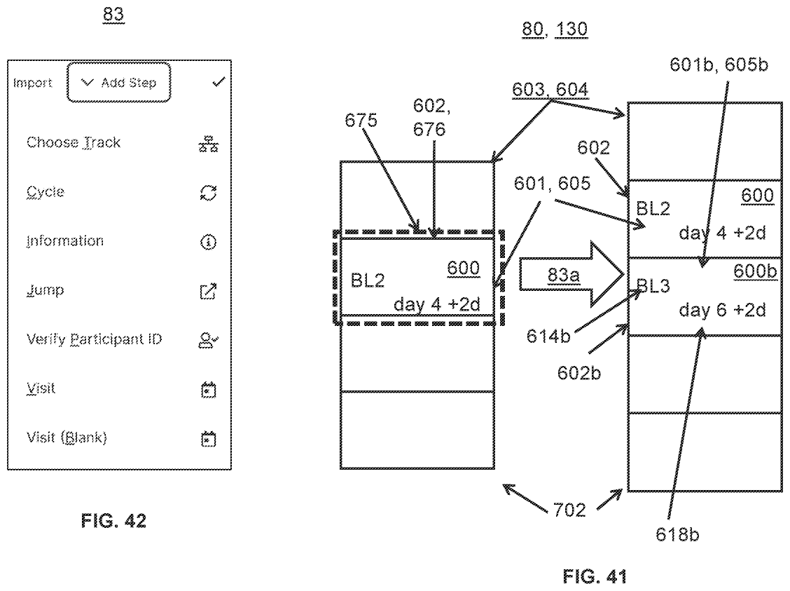

FIG. 41 illustrates an embodiment of Visit schedule interface 80 configured to enable using the visit schedule control 83 (illustrated by arrow 83a) to add a new node 600b to the visit schedule 130. New node 600b may be one of a new visit node, a new cycle node, a new jump node, and a new track selector node. A new node may be added in sequence with the last node, which may be a selected node 675, or the first or last node of a track. When a new node is added, the visit schedule interface 80 may be configured to automatically make the new node the currently selected node.

In response to using the visit schedule control 83 to add a new node 600b visit schedule interface 80 may be configured to receive a new node position 601b (e.g., new cycle position, new jump position, new visit position) and new node attributes 605b for the new node 600b from one or more of the input device, database 2, and server 3.

In a preferred embodiment, the new node position 601, comprising a new node track 603b and a new node step 602b, may be a system generated node position 601b based on the last node position 601. In this embodiment, new node track 603b may be the same as the last node track 603, and the new node step 602 may be system generated node step 602b successive to last node step 602. In other embodiments the new node tack and/or the new node step 602 may be provided by the input device, the database 2, a network transmission, and other methods. When the new node 600b is a new cycle node 600b, a new cycle start step 653 and a new cycle end step 652 may be determined (or generated) based on a selection position 676 of selected nodes 675 so that the new cycle start step precedes the selection start step 678 and the new cycle end step follows the selection end step 679.

The new node attributes 605b may comprise one or more of input node attributes 605b that have been provided externally to visit schedule interface 80 (through, e.g., input device, database 2, remotely), system generated node attributes 605b that have been determined (e.g., calculated) based on the last node attributes 605, and a combination of the foregoing. For example, the generated node attributes 605b may be determined from the last node attributes 605 by incrementing one or more of the last node attributes 605 (e.g., attributes of selected nodes 675) as appropriate so that the new node 602b is added in sequence with the last node 600, and using the remaining, non-incremented last node attributes 605 as generated node attributes 605b that are received as new node attributes 605b. For example, when adding a visit 610, the new visit id 614b (e.g., “BL3”) may be a generated visit id 614b in sequence with the last visit id 614 (e.g., “BL2”). In the same example, the new visit timing 618b (e.g., “day 6+2d”) is system generated visit timing 618 determined from the last visit timing 618 (e.g., “day 4+2d”) and it could be the next appropriate or available visit time window 619, or it could be based on the incremental period between previous visit time windows 619 (e.g., 2 days based on e.g., “day 4+1d” and “day 6+1d”) (sequentially follows the last visit timing 618 after adding fixed timing and/or plus/minus a flex period). After a new node is added to the visit schedule 130, the new node attributes 605 become the last node attributes 605 for that node type. In another example, if only one visit exists, attributes for the new visit will be automatically set based on the previous visit. If more than one previous visits exist, attributes for the new visit will be based on the trend seen in previous visits, so as if BL1 (day 14+/−1d) and BL2 (day 28+/−1d) exist and a new visit is added, new visit attributes will be automatically set to BL3 (day 42+/−1d).

In response to using the visit schedule control 83 to add a new node 600 for a track selector 627, visit schedule interface 80 may be configured to add to the visit schedule 130 one or more new destination tracks 634 based on the number of destination tracks 631 and destination track names 632 in the new track selector node attributes 630.

In response to using the visit schedule control 83 to add a new node, visit schedule interface 80 may be configured to cause GUI 43, servers 3, and/or database 2 to update the visit schedule 130 in the data 10 by inserting the new node in the step sequence 702 according to the new node position 601. The new node may be inserted in the data for example, by storing the new node (together with all node information, such as new node position 601, new node data id, new node attributes 605, etc.) in visit schedule 130 in database 2, and updating the data for the track, step sequence 702, and other relevant records, to indicate the addition of the new node and its position (e.g., tracks and steps). The node steps 602, node attributes 605, and other information of one or more nodes already in the visit schedule 130 may also be adjusted (e.g., incremented, reduced) to maintain the order of nodes in step sequence 702 following the addition of the new node. Update the node attributes 605 of each of the plurality of nodes (in the step sequence 702 based on the new node attributes 605.

When the new node is a new track selector node 627, visit schedule interface 80 may further cause the database 2 to update the visit schedule 130 in data by generating and storing data records for as many new destination tracks 634 as specified by the new track selector node attributes 630.

In response to using the visit schedule control 83 to add a new node, visit schedule interface 80 may be configured to update the track graphic to reflect the updated step sequence 702 with the new node. For example, the visit schedule interface 80 may display a new selectable node graphic visually representative of the new node and the new node attributes 605 in sequence with the selectable node graphics, and rearrange the selectable node graphics already displayed to maintain the visualization of the node order in the step sequence 702.

When the new node is a new track selector node 627, visit schedule interface 80 may further display new track graphics visually representative of the new destination tracks 634 specified by the new track selector node attributes 630.

Visit schedule interface 80 is configured to enable using the visit schedule control 83 to delete the selected nodes 675 from the visit schedule 130, by deleting the information associated with the selected nodes 675 from the data of the visit schedule 130 and updating the data for the step sequence 702 based on the selection position 676 (including e.g., the node track 603 and node step 602) to maintain the order of the remaining nodes in the step sequence 702. To update the step sequence 702, visit schedule interface 80 may, for example, cause the database 2 to adjust (e.g., by calculating and storing) each of the node steps 602 of the nodes that follow the selection end step 679. The visit schedule interface 80 may also be configured to adjust the node attributes 605 of the nodes in the visit schedule 130 to maintain appropriate sequence (e.g., decrease visit names 615 following deleting a visit) in the visit schedule 130. When a node is deleted, the visit schedule interface 80 may be configured to automatically make the node before or after the selected nodes 675 a new selected node.

When the deleted selected nodes 675 include a track selector node 627, the visit schedule interface 80 may be configured to further cause the database 2 to delete all data for the tracks associated with (e.g., created when adding) the deleted track selector node 627 and delete all nodes associated with the deleted tracks. If any of the deleted tracks splits into additional tracks, those additional tracks and their nodes may also be deleted.

In response to deleting the selected nodes 675, visit schedule interface 80 may be configured to update track graphic by removing from the display the selectable node graphics representative of the selected nodes 675 and rearranging the selectable node graphics to reflect the updated step sequence 702 following the deletion.

When the deleted selected node is a track selector node 627 visit schedule interface 80 may further remove the track graphic 750 for each track 700 connected with (e.g., generated by the addition of) the deleted track selector node 627, together with all selectable node graphics 753 of the track graphic.

Visit schedule interface 80 is configured to enable using the visit schedule control 83 to modify node attributes 605 of selected nodes 675. When the visit schedule control 83 is used to modify node attributes 605, visit schedule interface 80 may receive modified node attributes 605 for the selected nodes 675 from the input device, by programmatic instructions, from the database 2, remotely over a network, and by other methods. Visit schedule interface 80 may cause the database 2 to store the modified node attributes 605 replacing the modified node attributes 605 and updating visit schedule 130. Visit schedule interface 80 may also update the visual attributes of the selectable node graphics to be visually representative of modified node attributes 605.

Visit schedule interface 80 is configured to allow using the visit schedule control 83 to move the selected nodes 675 from their selection position 676 to a move to position 601 received by the visit schedule interface 80, illustrated as arrow 675A in FIG. 40. The move to position 601 may be received from the input device (e.g., typing, dragging the selection on the display, using keyboard shortcuts, etc.), from the GUI 43, from the database 2, and from other sources. The move to position 601 may comprise information about a move to track 603 and a move to node step 602. In response to receiving the move to position, visit schedule interface 80 causes the database 2 to update the visit schedule 130 by inserting the selected nodes 675 in the appropriate track and its step sequence 702 according to the move to position. To insert the selected nodes 675, the database 2 stores the move to track as part of node tracks 603 of the selected nodes 675 and stores move to node step 602 for each of the selected nodes 675 based on the move to step (e.g., determining each move to node step 602 for multiple selected nodes 675). Database 2 also may rearrange the step sequences 702 of the selected nodes 675 move from track 603 and the move to track 603 based on the move to position and the selection position 676 to maintain the sequence of nodes 600 in each of the step sequences 702. The move from track 603 and the move to track 603 may be the same as node track 603 of selected nodes 675, in which case the move from step sequence 702 and the move to step sequence 702 may also be the same. GUI 43 may also be configured to update the node attributes 605 of nodes in the move from track and move to track

To maintain the visualization of the visit schedule 130 when the selected nodes 675 are moved, visit schedule interface 80 may update one or more track graphics corresponding to the move from track and the move to track based on the selection position 676 and the move to position. Visit schedule interface 80 may reposition on the display the selectable node graphics associated with the selected nodes 675 by removing it from the display position representative of the selection position 676 and displaying it at a display position representative of the move to position. The visit schedule interface 80 may further rearrange the selectable node graphics 601 to reflect the moves selected nodes 675.

Visit schedule interface 80 is configured to retain in computer memory on in the database 2, information about a last node, which generally represent information about the last used node. The last node may have a last node position 601 comprising a last node step 602, and a last node track 603. The last node position 601 may be the position of the last node, and may be selection position 676, new node position 601, move to position 601, or a default node position 601. The last node may also comprise last node attributes 605 selected from the node attributes 605 of the selected nodes 675, the new node attributes 605, the modified node attributes 605, predetermined default node attributes 605, and combinations of the foregoing.

While the invention has been described with reference to exemplary embodiments, it will be understood by those skilled in the art that various changes, omissions, and/or additions may be made and equivalents may be substituted for elements thereof without departing from the spirit and scope of the invention. In addition, many modifications may be made to adapt a particular situation or material to the teachings of the invention without departing from the scope thereof. Therefore, it is intended that the invention is not limited to the embodiments disclosed as the best mode contemplated for carrying out this invention, but that the invention will include all embodiments falling within the scope of the appended claims. Moreover, unless specifically stated, any use of the terms first, second, etc., do not denote any order or importance, but rather the terms first, second, etc., are used to distinguish one element from another.

We claim:

1. A system for visualizing and managing a clinical study visit schedule, the system comprising:
a processor;
an input device;
a database storing data related to a clinical study, wherein the data comprise a study visit schedule comprising:
a track; and,
a plurality of nodes; and,
a visit schedule interface in data communication with the database and with the input device;
wherein each of the plurality of nodes comprises a node position and node attributes;
wherein the node position comprises a node track identifying the track, and a node step;
wherein the track comprises a step sequence of the plurality of nodes sequentially ordered according to the node step of each node;
wherein the track is selected from a study track, an exit track, and an unscheduled visits track;
wherein the visit schedule interface comprises a visit schedule control;
wherein the visit schedule interface receives the visit schedule from the database;
wherein the visit schedule interface is configured to display a track graphic visualizing the step sequence;
wherein the track graphic comprises a plurality of selectable node graphics;
wherein each of the selectable node graphics is associated with one node of the plurality of nodes;
wherein each of the selectable node graphics comprises visual attributes representative of the node attributes;
wherein the visit schedule interface is configured to enable using the visit schedule control to add a new node to the visit track;
wherein the visit schedule interface, responsive to using the visit schedule control to add a new node, is configured to update the track graphic by displaying a new selectable node graphic in sequence with the plurality of selectable node graphics;
wherein the visit schedule interface is configured to allow using the visit schedule control to delete one or more nodes of the plurality of nodes;
wherein the visit schedule interface, responsive to using the visit schedule control to delete one or more nodes, is configured to remove from the track graphic the selectable node graphics associated with the one or more nodes that were deleted;
wherein the visit schedule interface is configured to allow using the visit schedule control to move one or more nodes of the plurality of nodes to a move to position;
wherein the visit schedule interface, responsive to using the visit schedule control to move the one or more nodes, is configured to update the track graphic by repositioning the selectable node graphics of the one or more nodes that were moved to the move to position;
wherein the visit schedule interface is configured to allow using the visit schedule control to modify the node attributes of one or more nodes of the plurality of nodes, the new node attributes, and combinations thereof; and,
wherein the visit schedule interface, responsive to using the visit schedule control to modify the node attributes or the new node attributes, is configured to: (a) receive modified node attributes, and (b) update the visual attributes of the selectable node graphics to be visually representative of the modified node attributes.

2. The system of claim 1, wherein the visit schedule interface further comprises a selection control;
wherein the visit schedule interface is further configured to enable use of the selection control to select one or more nodes from the plurality of nodes as selected nodes by selecting one or more of the selectable node graphics;
wherein the selected nodes comprise a selection position;
wherein the visit schedule interface, responsive to using the visit schedule control to add a new node, is further configured to receive a new node position and new node attributes;
wherein the new node position is based on the selection position and causes the database to store the new node into the visit schedule and to insert the new node into the step sequence according to the new node position;
wherein the visual attributes of the new selectable node graphic are representative of the new node attributes;
wherein the visit schedule interface, responsive to using the visit schedule control to delete the selected nodes, is configured to (a) cause the database to delete the selected nodes from the visit schedule and to update the step sequence based on the selection position, and (b) remove from the track graphic the selectable node graphics associated with the selected nodes;
wherein the visit schedule interface, responsive to using the visit schedule control to move the node selection, is further configured to (a) receive the move to position, and (b) cause the database to store the node selection in the visit schedule and to update the step sequence based on the selection position and the move to position;
wherein the visit schedule interface is configured to allow using the visit schedule control to modify the node attributes of one or more nodes of the plurality of nodes, the new node attributes, and combinations thereof; and,
wherein the visit schedule interface, responsive to using the visit schedule control to modify the node attributes of the node selection, is configured to cause the database to store the modified node attributes replacing one or more of the node attributes of the selected nodes, and the new node attributes.

3. The system of claim 2, wherein the visit schedule interface further comprises an exit track control and one or more exit tracks;
wherein each exit track comprises an exit track name and exit reasons;
wherein the visit schedule interface displays one or more exit track graphics visually representative of the one or more exit tracks;
wherein the plurality of nodes are selected from the group consisting of a visit node, a cycle node, a jump node, a track selector node, an exit node, a participant verification node, and an information node;
wherein the visit schedule interface, responsive to using the visit schedule control to add the new node as a new track selector node, is configured to (a) receive new track selector node attributes comprising a number of destination tracks and destination track names, (b) cause the database to generate one or more new destination tracks based on the number of destination tracks, wherein each new destination track comprises a track name based on the destination track names, and to store the new destination tracks into the visit schedule, and (c) display new track graphics visually representative of the new destination tracks according to the new track selector attributes;
wherein the visit schedule interface is configured to receive from the exit track control one or more of (a) an indication to add one or more new exit tracks, (b) an indication to delete one or more of the exit tracks, and (c) a modified exit track name and modified exit reasons for the one more of the exit tracks;
wherein responsive to receiving the indication to add a new exit track the visit schedule interface is configured to (a) cause the database to generate and store the new exit track in the visit schedule, and (b) display a new exit track graphic visually representative of the new exit track;
wherein responsive to receiving the indication to delete one or more of the exit tracks the visit schedule interface is configured to (a) cause the database to delete the deleted exit tracks from the visit schedule, and (b) stop displaying the exit track graphics associated with the deleted exit tracks; and,
wherein responsive to receiving the modified exit track name and modified exit reasons the visit schedule interface is configured to (a) cause the database to store the modified exit track name and modified exit reasons in the visit schedule replacing the exit track name and the exit reasons, and (b) update the exit track graphic to be visually representative of the modified exit track name and modified exit reasons.

4. The system of claim 3, wherein the data further comprise a visit travel policy;
wherein the plurality of selectable node graphics are selected from a visit graphic, a cycle graphic, a jump graphic, a track selector graphic, an exit node graphic, a participant verification graphic, and an information graphic;
wherein the visit node comprises visit node attributes comprising a visit id, a visit name, a visit purpose, a visit study period, a visit timing, a visit duration, an allowed visit form, and a visit policy label;
wherein the visit policy label is associated with the visit travel policy;
wherein the cycle node comprises a cycle start step, cycle end step, a home track and cycle node attributes;
wherein the cycle node attributes comprise a cycle end condition and a cycle timing;
wherein the cycle end condition is selected from a manual termination and a number of repetitions;
wherein the jump node comprises a jump start position, a jump destination, and jump node attributes;
wherein the jump start position comprises a jump start track and a jump start step;
wherein the jump destination comprises a jump destination track and a jump destination step;
wherein the jump node attributes comprise a jump optional indicator;
wherein the track selector node comprises track selector node attributes comprising a number of destination tracks, destination track identifiers, and a track selection timing;
wherein the exit node comprises exit node attributes comprising an exit method selected from an exit study, a rollover to study, and a jump to exit track;
wherein the rollover to study comprises a clinical study identifier;
wherein the jump to exit track comprises a destination exit track;
wherein the jump graphic comprises a jump start graphic associated with the jump start position and a jump destination graphic associated with the jump destination;
wherein the cycle graphic comprises a cycle start graphic associated with the cycle start step and a cycle end graphic associated with the cycle end step; and,
wherein the track selector graphic comprises a track connector attribute visually connecting to the one or more new destination tracks.

5. The system of claim 4, wherein the node selection comprises a selection start step and a selection end step;
wherein the visit schedule interface, responsive to using the visit schedule control to add the new node as a cycle node, is configured to receive new cycle attributes, a new cycle start step, and a new cycle end step;
wherein the new cycle start step precedes the selection start step and the new cycle end step follows the selection end step;
wherein the visit schedule interface, responsive to using the visit schedule control to add the new node as a jump node, is configured to receive new jump attributes, a new jump start step, and a new jump destination step; and,
wherein the jump start step is in sequence with the selection start step, and the jump end step is in sequence with the selection end step.
